(19) United States
(12) Patent Application Publication
(10) Pub. No.: US 2025/0339443 A1
(43) Pub. Date: Nov. 6, 2025

(54) METHOD FOR TREATING OR PREVENTING CALCIUM RELEASE-ACTIVATED CALCIUM CHANNEL OR DISCOIDIN DOMAIN RECEPTOR 2 RELATED DISORDERS OR CONDITIONS

(71) Applicant: LumiSTAR Biotechnology, Inc., Taipei City (TW)

(72) Inventors: Yi-Chun Yeh, Miaoli County (TW); Yu-Fen Chang, Nantou County (TW)

(73) Assignee: LumiSTAR Biotechnology, Inc., Taipei City (TW)

(21) Appl. No.: 19/271,829

(22) Filed: Jul. 17, 2025

Related U.S. Application Data
(63) Continuation of application No. PCT/CN2024/073037, filed on Jan. 18, 2024.
(60) Provisional application No. 63/480,626, filed on Jan. 19, 2023.

(51) Int. Cl. A61K 31/538; A61K 31/122; A61P 11/00; A61P 13/12

(52) U.S. Cl. CPC A61K 31/538; A61K 31/122; A61P 11/00; A61P 13/12

(57) ABSTRACT

A method for treating or preventing a calcium release-activated calcium (CRAC) channel related disorder or condition and/or discoidin domain receptor 2 (DDR2) related disorders or conditions includes administering a subject in need thereof with a pharmaceutical composition. Also provided is a method for inhibiting CRAC channel activation and/or DDR2 activation in a cell of a subject, including administering the subject in need thereof with a pharmaceutical composition. The pharmaceutical composition includes an effective amount of at least one selected from the group consisting of WRG-28, atovaquone (Av), WRG-28 precursors, and atovaquone (Av) precursors; and a pharmaceutically acceptable carrier thereof.

### CROSS REFERENCE TO RELATED APPLICATIONS

This application is a continuation application of PCT Application No. PCT/CN2024/073037, filed on Jan. 18, 2024, which claims the benefit of U.S. Provisional Application No. 63/480,626, filed on Jan. 19, 2023. The contents of these applications are incorporated herein by reference.

### BACKGROUND OF THE INVENTION

### 1. Field of the Invention

The present disclosure relates to calcium release-activated calcium (CRAC) channel and discoidin domain receptor 2 (DDR2), particularly to a method for prevention or treatment of CRAC channel-related disorders or conditions and/or DDR2-related disorders or conditions.

### 2. Description of the Prior Art

The calcium release-activated calcium (CRAC) channel mediates Ca²⁺ influx, known as store-operated Ca²⁺ entry (SOCE). It protects against fibrosis by inhibiting transforming growth factor (TGF)-β1-induced epithelial-to-mesenchymal transition and fibroblast activation. The activation of the CRAC channel and discoidin domain receptor 2 (DDR2) is critical in myofibroblast activation and organ fibrosis. DDR2, on the other hand, as the CRAC channel downstream molecule, orchestrates collagen deposition, which is involved in cancer metastasis and organ fibrosis.

Fibrotic diseases pose global issues, particularly evident in aging societies. Infectious diseases-induced pulmonary fibrosis, cirrhosis, nephritis, diabetes or hypertension-induced chronic renal fibrosis, and cardiac fibrosis resulting from uremic cardiomyopathy are major leading causes of mortality and represent a significant burden on public health expenditure. Myofibroblasts play a critical role in organ fibrosis. Resident fibroblasts and pericytes constitute the primary sources of myofibroblasts, although other origins, such as macrophage, bone-marrow-derived cells, endothelial cells, have been suggested to contribute to the increasing myofibroblast population during disease progression; however, their role is still a subject of debate. Myofibroblasts are acknowledged for their great capability of extracellular matrix (ECM) production and remodeling, and α-smooth muscle actin (α-SMA) is hallmarked for not only myofibroblasts but also the diagnosis of fibrotic diseases.

Hypercytokinemia, also known as a cytokine storm, is an uncontrolled hyperinflammatory response resulting from a severe immune reaction spread from a localized inflammatory response to viral or bacterial infection. Immune hyperactivation in hypercytokinemia can occur due to various reasons: inappropriate triggering or danger sensing, initiating a response in the absence of a pathogen (e.g., in genetic disorders involving inappropriate inflammasome activation); inappropriate or ineffective amplitude of response, leading to excessive immune-cell activation (e.g., in CAR T-cell therapy); uncontrolled infections and prolonged immune activation (e.g., in Epstein-Barr virus, MERS-COV or SARS-COV-2 infections); or failure to resolve the immune response and return to homeostasis (e.g., in primary hemophagocytic lymphohistiocytosis).

The cytokine storm is considered the primary cause of the high mortality rate associated with COVID-19. Treatment with anti-inflammatory drugs such as corticosteroids (e.g., dexamethasone) or those targeting cytokine function (e.g., tocilizumab, an anti-interleukin 6 receptor antibody) has shown significant reductions in mortality among COVID-19 patients. Nevertheless, despite extensive use, two large randomized trials investigating tocilizumab did not demonstrate a survival benefit in hospitalized patients with COVID-19.

To date, there is no optimal therapeutic product specifically targeting either CRAC channel-related disorders, DDR2-related disorders, or both conditions. Consequently, there exists an unmet need to develop an effective pharmaceutical agent that ensures safety and tolerability in treating or preventing such conditions.

### SUMMARY OF THE INVENTION

Provided is a method for the treatment or prevention of CRAC channel-related disorder or condition and/or DDR2-related disorder or condition in a subject in need thereof. The method involves administering to the subject an effective amount/dose of WRG-28 and/or atovaquone (Av), or WRG-28 precursors and/or atovaquone (Av) precursors, along with pharmaceutically accepted carriers thereof.

The present disclosure also provided a use of the pharmaceutical composition of the present disclosure for treatment or prevention of a CRAC channel-related disorder or condition and/or a DDR2-related disorder or condition, comprising administering an effective amount of a pharmaceutical composition of the present disclosure to a subject in need thereof. The present disclosure additionally provides the pharmaceutical composition for use in treatment or prevention of a CRAC channel-related disorder or condition and/or a DDR2-related disorder or condition. Further provided is a use of the pharmaceutical composition for manufacture of a medicament for treatment or prevention of a CRAC channel-related disorder or condition and/or a DDR2-related disorder or condition.

In some aspects, the present disclosure provides a method for inhibiting CRAC channel and/or DDR2 activation in a cell of a subject, comprising administrating an effective amount/dose of WRG-28 and/or Av, or WRG-28 precursors and/or Av precursors, and pharmaceutically acceptable carriers thereof.

Also provided here is a use of the pharmaceutical composition of the present disclosure for inhibiting CRAC channel and/or DDR2 activation in a cell of a subject, comprising administering an effective amount/dose of WRG-28 and/or Av, or WRG-28 precursors and/or Av precursors, and pharmaceutically acceptable carriers thereof to the subject. The present disclosure additionally provides the pharmaceutical composition for use in inhibiting CRAC channel and/or DDR2 activation in a cell of a subject. Further provided is a use of the pharmaceutical composition for manufacture of a medicament for inhibiting CRAC channel and/or DDR2 activation in a cell of a subject.

In at least one embodiment, an effective amount/dose of WRG-28 and/or Av, or WRG-28 precursors and/or Av precursors, brings about a better effect on inhibiting TGF-β1-induced fibroblast activation, pericyte-to-myofibroblast differentiation, and/or TGF-β1-induced ECM remodeling.

In at least one embodiment, the effective amount/dose of WRG-28 and/or Av, or WRG-28 precursors and/or Av precursors shows a general effect in alleviating TGF-β1-induced myofibroblast activation.

In at least one embodiment, the effective amount/dose of WRG-28 and/or Av, or WRG-28 precursors and/or Av precursors in SOCE, shows varying capabilities in modulating the immune response to different extents.

In at least one embodiment, the effective amount/dose of WRG-28 and/or Av, or WRG-28 precursors and/or Av precursors in SOCE, significantly reduces the area positively stained with fibrillar collagen.

In at least one embodiment, the effective amount/dose of WRG-28 and/or Av, or WRG-28 precursors and/or Av precursors, shows potency in inhibiting tubulointerstitial fibrosis and exhibits protective effects against UUO-induced tubular atrophy and apoptosis.

In some embodiments, the effective amount/dose of WRG-28 and/or Av, or WRG-28 precursors and/or Av precursors, results in a decrease in the expansion of the fibrotic area and an increase in the expression of epithelial markers in mice.

In some embodiments of the present disclosure, the effective amount/dose of WRG-28 and/or Av, or WRG-28 precursors and/or Av precursors reverse fibrosis and promote tissue repair.

In some embodiments of the present disclosure, the effective amount/dose of WRG-28 and/or Av, or WRG-28 precursors and/or Av precursors, reduces myofibroblast activation and the subsequent reassembly of ECMs during renal fibrosis.

In some embodiments of the present disclosure, the effective amount/dose of WRG-28 and/or Av, or WRG-28 precursors and/or Av precursors, enhances tubular differentiation during renal fibrosis.

In at least one embodiment, the effective amount/dose of WRG-28 and/or Av, or WRG-28 precursors and/or Av precursors, shows profound effects in mitigating maladaptive repair, assisting tubular regeneration, and treating and protecting kidney function.

In at least one embodiment, the effective amount/dose of WRG-28 and/or Av, or WRG-28 precursors and/or Av precursors, brings a better effect in treating, preventing, and protecting the kidney from progressive fibrosis.

In some embodiments, the effective amount/dose of WRG-28 and/or Av, or WRG-28 precursors and/or Av precursors, shows a profound effect in reducing pulmonary fibrosis.

The present disclosure will no doubt become obvious to those of ordinary skill in the art after reading the following detailed description of the embodiment that is illustrated in the various figures and drawings.

### BRIEF DESCRIPTION OF THE DRAWINGS

FIG. 1A, FIG. 1B, FIG. 1C, FIG. 1D, FIG. 1E, FIG. 1F, FIG. 1G, FIG. 1H, FIG. 1I, FIG. 1J, FIG. 1K, FIG. 1L, FIG. 1M, and FIG. 1N show WRG-28 and atovaquone (Av) suppress the activation of DDR2 and calcium release-activated calcium (CRAC) channel. FIG. 1A indicates HEK293T cells were transiently overexpressed with DDR2 and then pretreated with different compounds (10 μM), including WRG-28, Av, donepezil hydrochloride (Dh), terazosin hydrochloride (Th), verapamil and flecainide for 30 minutes. Verapamil and flecainide were used as negative control here. After that, cells were treated with 50 μg/mL type I collagen for 12 hours. Cell lysates were harvested and subjected to Western blot analysis with antibodies against phosphotyrosine antibody (clone 4G10), DDR2, and β-actin. FIG. 1B shows the assessment of CRAC channel activity in HEK293T cells pretreated with 10 μM of BTP2, WRG-28, or Av while the cells were loaded with Fura 2-AM. CRAC channel activity was evaluated by applying 1 μM thapsigargin (Thap.) in a calcium-free solution to deplete the endoplasmic reticulum calcium store, followed by the reintroduction of 2 mM Ca²⁺ in an external solution. The concentration of cytosolic calcium ions increases as calcium flows through the CRAC channel, leading to the second peak in cytosolic calcium waves. Further analyses are displayed in FIG. 1C and FIG. 1D, where the second peak of each wave and the rate of entry were analyzed. Each bar represents the mean value derived from 47 to 103 cells. FIG. 1E displays the assessment of CRAC channel activity in NRK49F cells treated with 10 μM BTP2, WRG-28, or Av while the cells were loaded with Fura 2-AM. Further analyses are displayed in FIG. 1F and FIG. 1G, where the second peak of each wave and rate of entry were analyzed. Each bar represents the mean value derived from 83 to 176 cells. FIG. 1H displays the assessment of the effects of BTP2, WRG-28, or Av on CRAC channel activity in HK-2 cells. Further analyses are presented in FIG. 1I and FIG. 1J, where the second peak of each wave and rate of entry were analyzed. Each bar represents the mean value derived from 72 to 108 cells. The Orai1 and Stim1 puncta in HEK293T cells overexpressed with CFP-Orai1/Stim1-mCherry were assessed. Cells were pretreated with 10 μM DMSO (FIG. 1K), BTP2 (FIG. 1L), WRG-28 (FIG. 1M), or Av (FIG. 1N) for 30 min followed by stimulation with 1 μM thapsigargin for 15 min. Cells were then fixed and the puncta were observed under a confocal microscope (Olympus, FV-1000). In all the graphs, each bar represents mean±SEM and *, **, and *** denote p-value <0.05, 0.01, and 0.001, respectively.

FIG. 2A, FIG. 2B, FIG. 2C, FIG. 2D, FIG. 2E, FIG. 2F, FIG. 2G-1, and FIG. 2G-2 display how WRG-28 and Av suppress TGF-β1-induced fibroblasts activation and the differentiation of pericytes into myofibroblasts. FIG. 2A shows the assessment of TGF-β1-induced activation of renal fibroblasts, as determined by the upregulation of collagen 1a1 and α-SMA, in NRK49F cells treated with 10 μM DMSO, BTP2, WRG-28, or Av for 24 hours. FIG. 2B shows the relative protein levels of collagen 1a1 and α-SMA assessed from three independent experiments. Each bar represents the mean #SEM. FIG. 2C shows the assessment of TGF-β1-induced pericyte-to-myofibroblast activation in CCL-226 cells co-treated with 10 μM DMSO, BTP2, WRG-28, or Av for 24 hours. The protein levels of collagen 1a1 and α-SMA were assessed using Western blot analysis under various conditions. FIG. 2D shows NRK49F cells cultured in collagen gel and co-treated with 10 ng/ml TGF-1 and/or 10 μM DMSO, BTP2, WRG-28, or Av for 3 days. FIG. 2E shows gels that were released from the tissue culture plate, and the relative gel area was assessed after 8 hours of release. Each bar represents mean±SEM. FIG. 2F shows the assessment of TGF-β1-induced collagen alignment in NRK49F cells cultured in FITC-conjugated collagen gel and co-treated with 10 ng/ml TGF-β1 and/or 10 μM DMSO, BTP2, WRG-28, or Av for 5 days. The images were acquired using a confocal microscope (Olympus, MPE). FIG. 2G-1 and FIG. 2G-2 display the measurement of angles between collagen fibers and the cell membrane, conducted using Image J. At least five different images from each condition were analyzed. Each bar represents mean±SEM. In all graphs, *, **, and *** represent p-value <0.05, 0.01, and 0.001, respectively. ‘ns’ denotes p-value >0.05, indicating no significant difference.

FIG. 3A, FIG. 3B, FIG. 3C, FIG. 3D, FIG. 3E, FIG. 3F, and FIG. 3G demonstrate that WRG-28 and Av suppress the activation of TGF-β1-induced cardiac fibroblasts, pulmonary fibroblasts, and hepatic stellate cells. As shown in FIG. 3A, FIG. 3B, and FIG. 3C, the effects of BTP2, WRG-28, or Av on the activation of human cardiac fibroblast (HCF), human pulmonary fibroblast (MRC5), and rat hepatic stellate cells (HSC-T6) induced by TGF-β1 were assessed. Cells were co-treated with various compounds (10 μM) and TGF-β1 for 24 hours. Following this, cell lysates were harvested, and the protein expression was assessed by Western blot analysis. FIG. 3D depicts the examination of TGF-β1-induced fibronectin deposition and α-SMA expression in HCF treated with 10 μM of DMSO, BTP2, WRG-28, or Av through immunofluorescence staining. The images were captured using a confocal microscope, the Olympus FV-1000. FIG. 3E, FIG. 3F, and FIG. 3G illustrate the assessment of CRAC channel activity in HCF, MRC5, and rat HSC-T6. These cells were pretreated with 10 μM of BTP2, WRG-28, or Av for 45 min. The evaluation included measuring the second peak of each calcium wave and the rate of entry. Each bar within the figure represents the mean value derived from 77 to 91 cells for HCF, 31 to 49 cells for MRC5, and 43 to 94 cells for HSC-T6. In all the graphs, *, **, and *** denote p-values of <0.05, 0.01, and 0.001, respectively.

FIG. 4A, FIG. 4B, FIG. 4C, FIG. 4D, FIG. 4E, FIG. 4F, FIG. 4G, and FIG. 4H demonstrate how WRG-28 and Av reduce SOCE in T cells and macrophages, resulting in the downregulation of cytokine release. The effects of BTP2, WRG-28, Av, and CM-4620 on SOCE were evaluated in T cells (FIG. 4A) and macrophages (FIG. 4E). Cells were cultured on glass-bottom 96-well plates and pretreated with various compounds (10 μM) while being loaded with Fura 2-AM. Subsequently, the cytosolic calcium signal waves were recorded, and the second peak of each wave and rate of entry were assessed. Each bar represents the mean value derived from, 43 to 111 cells for T cells (FIG. 4A), and 52 to 91 cells for macrophages (FIG. 4E). Cytokine induction in T cells and macrophages was conducted as described in the methods section. The levels of cytokines, including IL-2, TNF-α, and IL-6 in the culture medium were assessed using ELISA kits for T cells (FIG. 4B, FIG. 4C, and FIG. 4D) and macrophages (FIG. 4F, FIG. 4G, and FIG. 4H), respectively. Each bar represents the mean±SEM. In all the graphs, *, **, and *** denote p-values of <0.05, 0.01, and 0.001, respectively.

FIG. 5A, FIG. 5B, FIG. 5C, FIG. 5D, FIG. 5E, FIG. 5F, FIG. 5G, FIG. 5H, FIG. 5I, FIG. 5J, and FIG. 5K demonstrate how WRG-28 and Av reduce renal fibrosis induced by unilateral ureteral obstruction (UUO). Surgical ligation was performed on the left ureter of 7-to-8-week-old male mice using a nylon surgical suture. Subsequently, the mice were treated with 5 mg/kg/day of DMSO (control), BTP2, WRG-28, or Av for 7 days. FIG. 5A displays the kidney tissue histology of treated mice, examined using hematoxylin and eosin (H&E) staining. FIG. 5B illustrates the evaluation of fibrillar collagen expression levels via Sirius red staining. FIG. 5C presents the measurement of the Sirius red-positive area using Image J. FIG. 5D, FIG. 5E, FIG. 5F, FIG. 5G, and FIG. 5H depict the protein levels of integrin β1, DDR2, Collagen 1a1, α-SMA, and E-cadherin in kidney samples from mice treated with 5 mg/kg/day of BTP2 (FIG. 5D), WRG-28 (FIG. 5F), or Av (FIG. 5H), which were assessed by Western blot analysis. The relative protein levels in mice treated with BTP2 (FIG. 5E), WRG-28 (FIG. 5G), or Av (FIG. 5I) were quantified using Image J. FIG. 5J displays the assessment of apoptotic cells via the TUNEL assay, presenting representative pictures. FIG. 5K illustrates the evaluation of apoptotic cells from 5-10 pictures in each condition.

FIG. 6A, FIG. 6B, FIG. 6C, FIG. 6D, FIG. 6E, FIG. 6F, and FIG. 6G demonstrate that treatments with WRG-28 and Av during obstruction promote tissue repair and reduce fibrosis after the obstruction is released. FIG. 6A illustrates a diagram depicting the timeline of operations and drug delivery. The left ureter was surgically ligated (UUO-L) for 7 days, after which it was re-connected to the bladder (RUUO-L). Various compounds, including DMSO (control), WRG-28, and Av, were intraperitoneally injected daily during ureter ligation (UUO-L) period. After following RUUO-L for an additional 13 days, the right ureter was ligated (UUO-R). Subsequently, mice were housed in metabolic cages on the day after surgery (day 21). On day 22, the left kidneys were removed for further analysis to evaluate kidney function. FIG. 6B shows the paraffin-embedded tissue sections that underwent hematoxylin and eosin (H&E) and Sirius red staining. FIG. 6C illustrates the measurement of the Sirius red-positive area in each group, conducted by using Image J from 5-10 images. The protein levels of integrin β1, collagen 1a1, DDR2, α-SMA, E-cadherin, SGLT2, NHE1, and β-actin were assessed in mice treated with WRG-28 (FIG. 6D) and Av (FIG. 6F) using Western blot analysis. Relative protein levels were quantified using Image J (FIG. 6E and FIG. 6G). Each bar represents mean±SEM. In all the graphs, *, **, and *** denote p-values of <0.05, 0.01, and 0.001, respectively.

FIG. 7A, FIG. 7B, FIG. 7C, FIG. 7D, FIG. 7E, FIG. 7F, and FIG. 7G demonstrate how WRG-28 and Av treatments reduce the obstruction-induced persistent progression of renal fibrosis after the obstruction is released. FIG. 7A shows a diagram illustrating the timeline of operations and drug delivery. The left ureter was surgically ligated (UUO-L) for 7 days, after which it was re-connected to the bladder (RUUO-L). Various compounds, including DMSO (control), WRG-28, and Av were intraperitoneally injected for 13 days after the release of left ureter ligation (during RUUO-L period). After 13 days of RUUO-L, the right ureter was ligated (UUO-R). Subsequently, the mice were housed in metabolic cages on the following day of surgery (day 21). On day 22, the left kidneys were removed for further analysis. FIG. 7B displays the paraffin-embedded tissue sections that underwent hematoxylin and eosin (H&E) and Sirius red staining. FIG. 7C illustrates the measurement of the Sirius red-positive area in each group using Image J from 5-10 images. The protein levels of integrin β1, collagen 1a1, DDR2, rotein levels of integrin 7F, and FB-actin were assessed in mice treated with WRG-28 (FIG. 7D) and Av (FIG. 7F) using Western blot analysis. The relative protein levels were measured and quantified using Image J (FIG. 7E and FIG. 7G). Each bar represents the mean±SEM. In all the graphs, *, **, and *** denote p-values of <0.05, 0.01, and 0.001, respectively. NS denotes a p-value >0.05, indicating no significant difference between the two groups.

FIG. 8A, FIG. 8B, FIG. 8C, FIG. 8D, FIG. 8E, FIG. 8F, FIG. 8G, and FIG. 8H illustrate how WRG-28 and Av restore epithelial differentiation and reduce collagen deposition and myofibroblast expansion in RUUO-induced persistent progression of renal fibrosis. The frozen tissue blocks were utilized for immunofluorescence staining. FIG. 8A displays the expression of laminin alpha 1 and collagen 1a1 in mouse kidneys treated with DMSO, WRG-28, or Av during ureter ligation (7*/15d) or after the obstruction was released (7/15*d). Laminin alpha 1 and collagen 1a1 were detected using primary antibodies, followed by secondary antibodies conjugated with Alexa-488 (green) and Alexa-594 (red), respectively. Hoechst 33258 (blue) was used for labeling nuclei. FIG. 8A-(ii) illustrates the fluorescent intensity of collagen 1a1 measured using Image J. Analysis was conducted on 5-10 areas from each of the 5 pictures, with each bar representing the mean±SEM. In FIG. 8A-(i), the thickness of laminin alpha 1 was measured using Image J by analyzing 5-10 areas from each of the 5-10 pictures. The median is marked by a solid line, while the quartiles are indicated by dotted lines. FIG. 8B displays α-SMA marked by an antibody labeled with Cy3 (red), while Hoechst 33258 (blue) was used for labeling nuclei. FIG. 8B-(iii) illustrates the fluorescent intensity of α-SMA, measured using Image J. Quantification was conducted on at least 5 areas from each of the 5 pictures, with each bar representing the mean±SEM. FIG. 8C displays the detection of Laminin alpha 1 and SGLT2 using primary antibodies, followed by secondary antibodies conjugated with Alexa-594 (red) and Alexa-647 (cyan), respectively. Additionally, Hoechst 33258 (blue) was used for labeling nuclei. FIG. 8C-(iv) illustrates the quantification of apical membrane expression of SGLT2 in SGLT2 positive cells for each condition. Analysis was conducted on at least 5 pictures from each condition, with each bar representing the mean±SEM. FIG. 8D displays the detection of DDR2 and AQP1 using primary antibodies, followed by secondary antibodies conjugated with Alexa-488 (green) and Alexa-594 (red), respectively. Hoechst 33258 (blue) was used to label nuclei. The expressions of DDR2 (FIG. 8D-(v)) and AQP1 (FIG. 8D-(vi)) were analyzed and quantified based on 5-8 pictures from each condition. Each bar in the figures represents the mean±SEM. Furthermore, the cell percentage exhibiting membrane expression of AQP1 was also analyzed and quantified (FIG. 8D-(vi)). FIG. 8E displays the examination of apoptotic cells in mice that received treatment with different compounds during obstruction (7*/15d) through the TUNEL assay. Additionally, Ki67-positive cells, representing proliferating cells, were detected via immunohistochemistry using specific antibodies in mice subjected to various compound treatments during obstruction (7*/15d). FIG. 8F illustrates the quantification of apoptotic and proliferating tubular cells, conducted using at least 10 pictures from each condition. Each bar in the figure represents the mean±SEM. FIG. 8G displays the examination of apoptotic cells in mice that received various compound treatments after the obstruction was released (7/15*d) through the TUNEL assay. Furthermore, Ki67-positive cells, indicative of proliferating cells, were identified using specific antibodies in mice subjected to different compound treatments after the obstruction was released (7/15*d). FIG. 8H illustrates the quantification of apoptotic and proliferating tubular cells, with data collected from at least 10 pictures from each condition. Each bar within the graph represents the mean±SEM. In all the graphs, *, **, and *** denote p-values of <0.05, 0.01, and 0.001, respectively.

FIG. 9A and FIG. 9B depict the effects of WRG-28 and Av treatments on the improvement of kidney function following obstruction-induced kidney injury. Blood samples were collected from mice subjected to DMSO, WRG-28, or Av treatment during renal obstruction (7*/15d) or after the obstruction was released (7/15*d). The levels of blood urea nitrogen (BUN) are displayed in FIG. 9A, and creatinine (CRE) levels are shown in FIG. 9B. Each bar represents the mean #SEM. In the graphs, *, **, and *** denote p-values <0.05, 0.01, and 0.001, respectively.

FIG. 10A and FIG. 10B display how WRG-28 and Av treatments effectively reduce RUUO-induced overwhelming fibrosis and inflammation. In FIG. 10A, total ribonucleic acid (RNA) extracts from control mice (sham) and mice treated with DMSO, WRG-28, or Av after the obstruction was released (7/15*d) underwent ingenuity pathway analysis (IPA). The genes obtained are listed in FIG. 10B. Both FIG. 10A and FIG. 10B illustrate large-scale transcriptome analysis, further affirming the potent effects of WRG-28 and Av in preventing and protecting kidneys from progressive fibrosis.

FIG. 11A, FIG. 11B, FIG. 11C, and FIG. 11D display how treatment with WRG-28 or Av effectively reduces pulmonary fibrosis. A Bleomycin (BLM)-induced lung fibrosis mouse model was used, and the timelines of operation and drug delivery are depicted in FIG. 11A. FIG. 11B displays the evaluation of lung function across various indices. The hydroxyproline content, serving as a marker of collagen deposition, was extracted from the right lobe and analyzed; the results are shown in FIG. 11C. FIG. 11D depicts histological analysis derived from the left lobe, quantifying the fibrotic area through staining with Masson's Trichrome and Picrosirius red, respectively.

### DETAILED DESCRIPTION

The following embodiments are provided to illustrate the present disclosure in detail. A person having ordinary skills in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

In order that the present invention may be more readily understood, certain terms are first defined. In addition, it should be noted that whenever a value or range of values of a parameter are recited, it is intended that values and ranges intermediate to the recited values are also intended to be part of this invention.

As used herein, the singular forms “a,” “an,” and “the” include plural referents, unless expressly and unequivocally limited to one referent. For example, “an element” means one element or more than one element, e.g., a plurality of elements. The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “administering” or “administration” refers to the placement of an active agent into a subject by a method or route which results in at least partial localization of the active agent at a desired site to produce a desired effect. The active agent described herein may be administered by any appropriate route known in the art.

The numeral ranges used herein are inclusive and combinable, any numeral value that falls within the numeral scope herein could be taken as a maximum or minimum value to derive the sub-ranges therefrom. For example, it should be understood that the numeral range “0.1 to 10 μM” comprises any sub-ranges between the minimum value of 0.1 μM to the maximum value of 10 μM, such as the sub-ranges from 0.1 μM to 5 μM, from 1.0 μM to 10 μM, from 0.5 μM to 8 μM and so on. In addition, a plurality of numeral values used herein can be optionally selected as maximum and minimum values to derive numerical ranges. For instance, the numerical ranges of 0.1 μM to 5 μM, 0.1 μM to 10 μM, and 5 μM to 10 μM can be derived from the numeral values of 0.1 μM, 5 μM, and 10 μM.

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

As used herein, “subject” is used to mean any vertebrate including, but not limited to, humans, or non-human mammals such as deer, mule, elk, or mule deer, seeking to improve a condition, disorder, or disease, including CRAC channel- and/or DDR2-related disorders or conditions, e.g., organ fibrosis, hypercytokinemia (i.e., cytokine storm), cancers and COVID-19. However, advantageously, the subject is a mammal such as a human, or an animal mammal such as a domesticated mammal, e.g., a dog, a cat, a horse, a rat, a mouse, or the like.

Serum cytokines, including interleukin-1β (IL-1β), interleukin-2 (IL-2), interleukin-6 (IL-6), TNF (tumor necrosis factor), interferon-γ (IFN-γ), macrophage inflammatory protein (MIP) 1α and MIP 1β, are elevated in individuals with a cytokine storm. In one embodiment of the present disclosure, cytokine is selected from the group consisting of any one of interleukin 1 to interleukin 36, a tumor necrosis factor alpha, a tumor necrosis factor (TNF) α, a CD40 ligand, a Fas ligand, a tumor necrosis factor-related apoptosis inducing ligand, and a tumor necrosis factor superfamily member 14, and any combination thereof. In one embodiment of the present disclosure, the cytokine is an interleukin 2, an interleukin 6, or the tumor necrosis factor (TNF) α. Elevated cytokines can result in endothelial dysfunction, vascular damage, and paracrine/metabolic dysregulation, consequently causing damage to multiple organ systems. During the early state of hypercytokinemia, the elevation of acute-response cytokines, like TNF and IL-1β, as well as chemotactic cytokines, such as IL-8 and MCP-1, contributes to a sustained increase in IL-6. IL-6 is considered one of the more complex cytokines due to its production by and action on both immune and non-immune cells across multiple organ systems. IL-6 plays a crucial role in a cytokine storm, contributing to processes such as neutrophil chemotaxis and lymphocyte necrosis. Blocking upstream events associated with the cytokine response, such as inhibiting macrophage signalling to decrease IL-6 production or T-cell activity to lower cytokine levels, may present a potential therapeutic target for managing the cytokine storm. In some embodiments of the present disclosure, the cytokine is selected from the group consisting of a chemokine, an interferon, an interleukin, a lymphokine, and a tumor necrosis factor.

In one embodiment of the present disclosure, the CRAC channel-related disorder or condition and/or DDR2-related disorder or condition are selected from the group consisting of a cytokine storm syndrome, a fibrotic disorder, cancer, arthritis, a cardiorespiratory disease, an inflammatory disease, an autoimmune disease, an inflammatory bowel disease (IBD), an allergic disease, an acute kidney injury (AKI), a chronic kidney disease (CKD), uremic cardiomyopathy, nephrogenic systemic fibrosis (NSF), cystic fibrosis, polycystic kidney disease (PKD), pulmonary fibrosis, and any combination thereof.

In one aspect of the present disclosure, the treatment or prevention of the CRAC channel-related disorder or condition and/or DDR2-related disorder or condition comprises the inhibition of the CRAC channel activation. In another aspect of the present disclosure, the treatment or prevention of the CRAC channel-related disorder or condition and/or DDR2-related disorder or condition comprise the inhibition of DDR2 activation. In a further aspect of the present disclosure, the treatment or prevention of the CRAC channel-related disorder or condition and DDR2-related disorder or condition comprises the inhibition of both CRAC channel and DDR2 activation. In one embodiment of the present disclosure, the treatment or prevention of cytokine storm syndrome comprises the reduction of SOCE in T cells and macrophages. In at least one embodiment, the WRG-28, the atovaquone, the WRG-28 precursors, or the atovaquone precursors inhibit CRAC channel activation, DDR2 activation, SOCE, and/or cytokine expression.

In some embodiments of the present disclosure, the cytokine storm syndrome is an infection-induced cytokine storm syndrome. In other embodiments of the present disclosure, the cytokine storm syndrome is triggered by COVID-19. In some embodiments of the present disclosure, the cytokine storm syndrome is triggered by the pathogens selected from influenza virus, Epstein-Barr virus (EBV), severe acute respiratory syndrome coronavirus (SARS-COV), Middle East respiratory syndrome coronavirus (MERS-COV), and SARS-COV2.

In one embodiment of the present disclosure, the fibrotic disorders are tissue fibrosis or organ fibrosis. In another embodiment of the present disclosure, the fibrotic disorders are cardiac fibrosis, pulmonary fibrosis, liver fibrosis, nephritis, diabetes, renal fibrosis, kidney fibrosis, or any combination thereof. In some embodiments of the present disclosure, the fibrotic disorders are infection-induced fibrotic disorder, obstruction-induced fibrotic disorder, or drug-induced fibrotic disorder. In at least one embodiment of the present disclosure, the infection-induced fibrotic disorders are COVID-19-induced fibrotic disorders. In at least one embodiment of the present disclosure, the obstruction-induced fibrotic disorders are ureteral obstruction-induced kidney fibrosis.

In at least one embodiment, the treatment or prevention of the fibrotic disorder improves a kidney function, a pulmonary function, a liver function, a cardiac function; promotes a tissue repair, an epithelium differentiation; and inhibits a collagen deposition, a myofibroblast expansion, and/or a TGF-β-associated fibroblast activation. In at least one embodiment, the TGF-β-associated fibroblast activation is a TGF-β1-associated fibroblast activation. In at least one embodiment, the treatment or prevention of the fibrotic disorder comprises the promotion of tissue repair, restoration of epithelium differentiation, reduction of collagen deposition, suppression of myofibroblast expansion, and/or suppression of TGF-β1-associated fibroblast activation in the fibrotic disorders or conditions.

In at least one embodiment, the treatment or prevention of fibrotic disorders improves the kidney function. In some embodiments, the treatment or prevention of the fibrotic disorders improves the pulmonary function. In other embodiments, the treatment or prevention of the fibrotic disorders improves the liver function and/or cardiac function.

In some embodiments, the cancer is melanoma, or carcinoma of the head and neck, brain, nervous system, thyroid, thymus, esophagus, stomach, lung, breast, gastrointestinal tract, colon and rectum, liver, pancreas, kidney, adrenal cortex, genitourinary system, prostate, bladder, urothelium, uterus, cervix, ovary, skin, or hematologic malignancy. In at least one embodiment, the cancer is small cell lung cancer (SCLC), non-small cell lung cancer (NSCLC), squamous carcinoma of lung or adenocarcinoma of lung. In other embodiments, the cancer is primary cancer or secondary cancer. In further embodiments, the cancer is localized cancer, regional cancer, advanced cancer, or metastatic cancer. In at least one embodiment, the cancer is solid tumor or non-solid tumor. In other embodiments, the cancer is sarcoma, carcinoma, lymphoma, or leukemia.

### Materials and Methods

### Cell Culture and Treatment

The human embryonic kidney cells (HEK293T), rat kidney fibroblasts (NRK49F), human cardiac fibroblasts (HCF), human pulmonary fibroblast (MRC5), and rat liver stellate cells (HSC-T6) were purchased from ATCC (via the United Kingdom supplier LGC). The mouse pericyte cell line, CCL-226, is a kind gift from Professor S-L. Lin (National Taiwan University). Cells were maintained in Dulbecco's modified Eagle's medium (DMEM) (Thermo Scientific) containing 10% fetal bovine serum (Invitrogen) and 1% penicillin-streptomycin followed the manufacturer's instructions. Jurkat T cells and human monocytic THP-1 cells were cultured in Roswell Park Memorial Institute medium (RPMI 1640, Gibco) culture medium containing 10% of fetal bovine serum (Invitrogen) and 1% penicillin, and 1% streptomycin.

In TGF-β1-induced cell differentiation, cells were seeded on a type I collagen-coated tissue culture dish and then treated with different compounds for 4 hours. Cells were then treated with 10 ng/ml TGF-β1 for another 24 hours before protein analysis or immunocytochemistry study.

Different chemical compounds were used in the studies including BTP2 (Millipore, CAS 223499-30-7), WRG-28 (MCE #HY-114169), atovaquone (Av) (Cayman #23802), donepezil hydrochloride (Dh) (Millipore, CAS 120011-70-3), and terazosin hydrochloride (Th) (Millipore, CAS 70024-40-7). Av, donepezil hydrochloride (Dh), and terazosin hydrochloride were structurally comparable to BTP2 and WRG-28 by compound-database screening.

### Plasmids and Transfection

The DDR2 and CFP-Orai1/Stim1-mCherry were overexpressed in HEK293T cells by using Lipofectamine 2000 (Invitrogen) with 200 ng of plasmid DNA. The DDR2 plasmid is a kind gift from professor B. Leitinger (University College London, UK). CFP-Orai1 and Stim1-mCherry is a kind gift from W-T. Chiu (National Cheng-Kung University).

### Cytosolic Ca2+ Imaging

Cells cultured on a glass-bottom 96 well plate for 48 hours were treated with different compounds (10 μM) and Fura 2-AM (1 μM) simultaneously in a solution containing 145 mM NaCl, 2.8 mM KCl, 2 mM CaCl2, 2 mM MgCl2, 10 mM D-glucose, 10 mM HEPES, pH 7.4 for 40 minutes in the dark. Cells were then washed and incubated with the same solution in addition to the supplement of Fura 2-AM for another 15 minutes for fully de-esterification. Ca2+-free solution (145 mM NaCl, 2.8 mM KCl, 2 mM MgCl2, 10 mM D-glucose, 10 mM HEPES, 0.1 mM EGTA, pH 7.4) was applied to cells prior to image. 1 μM thapsigargin was added to induce endoplasmic reticulum (ER) calcium store depletion followed by applying 2 mM calcium solution to assess the activation of calcium release-activated calcium (CRAC) channels. Cells were alternately excited at 340 and 380 nm, and images were acquired every 2 seconds. Cytosolic calcium signals were represented by the 340 nm/380 nm ratio (R). All the images were analyzed by using IGOR Pro software.

### Western Blot Analysis

Cell lysates were harvested in RIPA buffer (150 mM NaCl, 1 mM EGTA, 50 mM Tris pH 7.4, 10% glycerol, 1% Triton X-100, 1% sodium deoxycholate, 0.1% SDS, and protease inhibitor cocktail) and collected from culture dish with cell scrapers. For tissue sample preparation, half of the kidney tissues were cut into ˜1 mm3 and then put into 500 μl RIPA buffer in 2 ml tubes. Mechanical homogenizers by agitating beads at high speeds for 30 seconds were used for tissue protein extraction. Protein samples in supernatants were collected after centrifuging the tubes at 10000 g for 10 min at 4° C. 20-30 μg protein samples were resolved by 7.5% or 10% SDS-PAGE. The protein expression levels were assessed by specific primary antibodies, including antibodies against phosphotyrosine (clone 4G10; Millipore), DDR2 (R&D), α-SMA (Sigma), Collagen 1a1 (Boster), E-cadherin (BD Biosciences), SGLT2 (Proteintech), NHE1 (Novus) and β-actin (Clone C4, Millipore), followed by incubating with secondary antibodies conjugated with HRP and detected by ECL kit (Thermo Scientific).

### Gel Contraction and Collagen Alignment Assay

The collagen gel was made by mixing 3 ml rat tail collagen (Corning), 1 ml 5.7×DMEM, 500 μl 2.5% NaHCO3, 1 ml 0.1 M HEPES, 100 μl 0.17 M CaCl2, 100 μl 1 N NaOH, and 4.3 ml culture medium containing 2×10⁶ NRK49F cells. 2 ml medium with or without 10 ng/ml TGF-β1 was applied onto gel in each well of 6 well-plate and cells were incubated for 3 days. After that gels were released from the culture plate and the gel area was quantified by Image J.

FITC-conjugated collagen was used to make collagen gel and cells were grown in the gel for 5 days. After that cells were fixed with 4% paraformaldehyde and phalloidin-TRICT was used as counterstaining in the evaluation of the cell body. The angles between fibers and cell membrane were measured by image J.

### Cytokine Induction and Assessment

THP-1 monocytes were differentiated into MI macrophages by incubation with 0.1 μg/mL phorbol 12-myristate 13-acetate (PMA, Sigma, P8139) for 24 h, followed by incubation with 20 ng/ml of IFN-γ (MCE, HY-P7025) and 1×LPS (Thermo, 00-4976-93) for another 3 days. Different dosages of drugs were co-treated with the induction medium, containing 20 ng/ml of IFN-γ (MCE, HY-P7025) and 1×LPS (Thermo, 00-4976-93). Jurkat T cells were treated with 1 μg/mL PHA and 1 μg/mL PMA for 24 hrs, followed by being pre-treated with different dosages of studied compounds for 30 mins prior to the cytokine induction.

The supernatants were harvested at indicated time points, and the cytokines, including IL-2, IL-6, and TNF-α, were conducted by ELISA kits following the manufacturer's protocol. The ELISA kits that were used in the experiments were listed, ELISA MAX™ Deluxe Set Human IL-2 (BioLegend, Cat. no. 431804), Human IL-6 ELISA MAX™ Deluxe (BioLegend, Cat. no. 430504), and ELISA MAX™ Deluxe Set Human TNF-alpha (BioLegend, Cat. no. 430204). Absorbance was measured at 450 nm with a microplate reader (SpectraMax iD3, USA).

### Immunofluorescence Staining

Cells cultured on chamber slides were fixed with 4% paraformaldehyde for 10 minutes followed by a permeabilization step by incubating cells in 0.5% Triton X-100 in PBS for 5 minutes. Cells were then incubated in SuperBlock blocking buffer (Thermo Scientific) for 1 h at room temperature. The protein expression was detected by incubating cells with specific primary antibodies followed by incubating cells with secondary antibodies against mouse or rabbit IgG conjugated with Alexa-488 or -594 nm (Invitrogen), respectively. Hoechst 33258 (10 μg/ml) is used for nuclei staining. All images were visualized and taken by confocal microscopy (Olympus, FV-1000).

Tissue slides in 15 μm-thickness from Tissue-Tek OCT compound embedded tissue block were used for immunofluorescence staining. Tissues were fixed and permeabilized by immersing in ice-cold acetone for 10 minutes followed by incubating with SuperBlock blocking buffer (Thermo Scientific) at room temperature for 1 h to minimize the background signals. The protein expression and localization were detected by specific primary antibodies, including antibodies against collagen 1a1 (Boster), Laminin alpha 1 (R&D), SGLT2 (Proteintech), AQP1 (Novus), DDR2 (R&D systems), and α-SMA (Sigma), followed by labeling with secondary antibodies conjugated with a fluorescent protein (Invitrogen). The images were taken by confocal microscopy (Olympus, FV-1000).

### Unilateral Ureteral Obstruction (UUO) and Reversible Unilateral Ureteral Obstruction (RUUO)

Procedures involving animal subjects have been approved by the Institutional Animal Care and Use Committee (IACUC) at the National Laboratory Animal Center. 7-8 weeks old male C57BL/6 mice were obtained from the animal center. Unilateral ureteral obstruction (UUO) was performed by ligating the left ureter and mice were treated with 5 mg/kg/day DMSO, BTP2, WRG-28, or Av for 7 days after ligation. After 7 days, mice were sacrificed and the kidneys were removed and used for analysis. One-quarter of the kidney was fixed by 4% paraformaldehyde and embedded in paraffin or one-quarter of the kidney was embedded in the Tissue-Tek OCT compound. Half of the kidneys are used for protein analysis. In the reversible unilateral ureteral obstruction (RUUO) experiment, the ligated-left ureter was re-connected to the bladder after UUO surgery for 7 days. The compounds, WRG-28 and Av, were injected during ligation (7*/15d) or after ligation was released (7/15*d). To assess the kidney function, the right ureter was ligated the day before leaving mice in the metabolic cage. The urine and blood were collected for blood urea nitrogen (BUN) and creatinine (CRE) test.

### Hematoxylin and Eosin (H&E) Staining

Deparaffinize and rehydrate tissue slides were immersed in filtered Harris Hematoxylin for 10 seconds. After extensively washing in tap water, samples were immersed in Alcoholic-Eosin for 2 minutes. The tissue was then dehydrated and mounted by resinous medium and glass coverslips.

### Sirius Red Staining

Deparaffinize and rehydrate tissue slides were stained with Picrosirius red for one hour, followed by an extensive wash with acidified water. After dehydration through 3 runs of change of 95% ethanol, 2 runs of change of 100% ethanol, and 3 runs of change of xylene, tissue slides were mounted by resinous mounting medium and glass coverslips. The tissue was then dehydrated and mounted by resinous medium and glass coverslips.

### Immunohistochemistry

Deparaffinize and rehydrate tissue slides were subjected to the procedure for antigen retrieval by using citrate buffer and heated by microwave. 3% H₂O₂ in methanol was used to block endogenous peroxidase activity and SuperBlock blocking buffer (Thermo Scientific) was used to minimize the background signals. Following the blocking step, tissue slides were blotted with anti-Ki67 primary antibody and the antibody against mouse IgG conjugated with horseradish peroxidase (HRP). HRP signal was then detected by diaminobenzidine (DAB). Hematoxylin was used as counterstaining in the evaluation of the nucleus. The tissue was then dehydrated and mounted by resinous medium and glass coverslips.

TUNEL (Terminal Deoxynucleotidyl Transferase dUTP Nick and Labeling) Assay

The TUNEL assay kit was purchased from Abcam (ab206386). The procedure was carried out following the manufactory's instructions. In brief, the deparaffinized and rehydrate tissue slides were incubated in proteinase K solution for 20 minutes, 3% H₂O₂ for 5 min, and then the TdT enzyme and reaction mix for 1.5 hours at 37° C. The signal was then detected and developed by DAB solution. Methyl green was used for counterstain in evaluating the healthy cells. Tissue slides were then dehydrated and mounted by resinous medium and glass coverslips.

### 5/6 Nephrectomy

7-8 weeks old male C57BL/6 mice were used for the experiment. The upper and lower poles of the left kidney were ligated, and the right kidneys were excised 1 week later. After 4 weeks, mice have received 5 mg/kg/day of DMSO, WRG-28, or Av injection through peritoneum for the following 4 weeks. Urine and blood were collected for blood urea nitrogen (BUN) and creatinine (CRE) tests, and the kidneys were subjected to protein and histology analysis.

### Blood Urea Nitrogen and Creatinine Detection

The content of BUN and CRE in serum and urine were detected by an automated clinical chemistry analyzer (Fuji, Dri-Chem 4000i). All procedures followed the manufactory's instructions.

### Next-Generation Sequencing-Based RNA Sequence and Analysis

Total RNA was extracted from kidneys by homogenizing the samples in TRIzol reagent (Invitrogen). Chloroform was added and the homogenate separates into three layers, the upper clear aqueous containing RNA was transferred into a new tube, and RNA was precipitated by adding isopropanol. The pellets were washed with 70% ethanol and then dissolved in RNase-free water. The RNA passes the quality control, including RNA integrity number (RIN) >7, OD260/OD280 ˜1.8, etc., were sent for sequencing. Transcriptomes were analyzed by ingenuity pathway analysis (IPA).

### Bleomycin-Induced Lung Fibrosis Model

8-to-10-week-old male WT (C57BL/6) mice received intratracheal instillation of bleomycin (bleocin; Nippon Kayaku) at the dose of 3 mg/kg diluted in PBS or PBS only (sham). To study fibrosis, lung samples were collected on day 21 for further analysis.

### Lung Function Tests

Lung function was assessed using the flexi Vent system (Scireq, Montreal, QC, Canada). Mice were tracheostomized and ventilated at a rate of 150 breaths/min, tidal volume of 10 ml/kg, and a positive end-expiratory pressure (PEEP) of 2-3 cmH2O. A deep inflation perturbation was used to estimate the inspiratory capacity (IC). Pressure-Volume loops were generated by constant increasing pressure, followed by regular decreasing pressure. Other lung function parameters like resistance, compliance, and elastance were measured by using SnapShot-150.

### Hydroxyproline Assay

Levels of hydroxyproline in lung tissues were measured using the Hydroxyproline Colorimetric Assay kit (BioVision, Milpitas, CA, USA). Briefly, 10 mg of frozen right middle lobes homogenized in 100 μL of hydrochloric acid (HCl, 12 N), and hydrolyzed at 120° C. for 3 h. Then, 10 μL of individual samples were used to quantify the absorbance at 560 nm. Hydroxyproline content was presented in μg per lobe.

### Histology

Mouse left lungs were fixed overnight in 4% paraformaldehyde (PFA), embedded in paraffin, and sectioned in 5 μm thickness for picrosirius red (Abcam, Cambridge, UK) and Masson's trichrome staining (Sigma Aldrich, MO, USA). Measurement of fibrotic area was quantified using ImageJ software (NIH, http://rsbweb.nih.gov/ij/).

### Statistical Analysis

All results were expressed as means±SEM. Two-tailed Student t-test was used to compare differences between two groups in all the experiments. GraphPad Prism was used, and statistical significance was set at a P-value of <0.05. In all the graphs, *, **, and *** denote P-value <0.05, 0.01, and 0.001, respectively.

### EXAMPLES

Exemplary embodiments of the present disclosure are further described in the following examples, which should not be construed to limit the scope of the present disclosure.

### Example 1. WRG-28 and Av Inhibit the Activation of DDR2 and SOCE

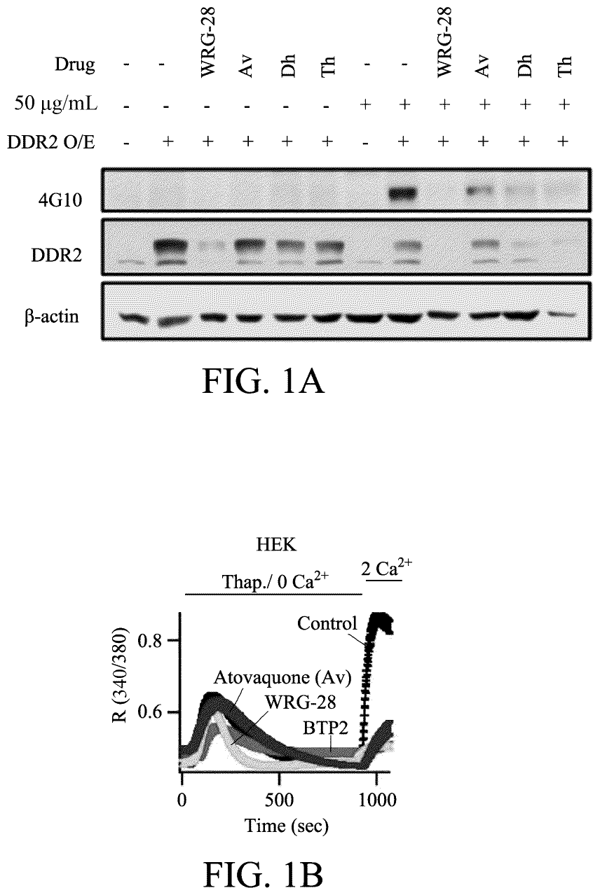

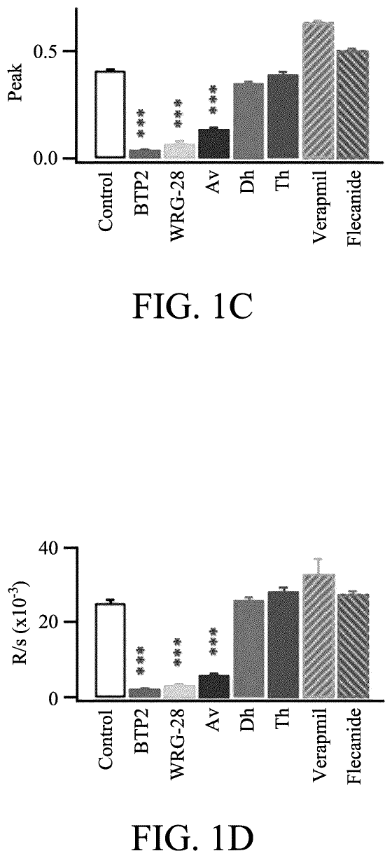

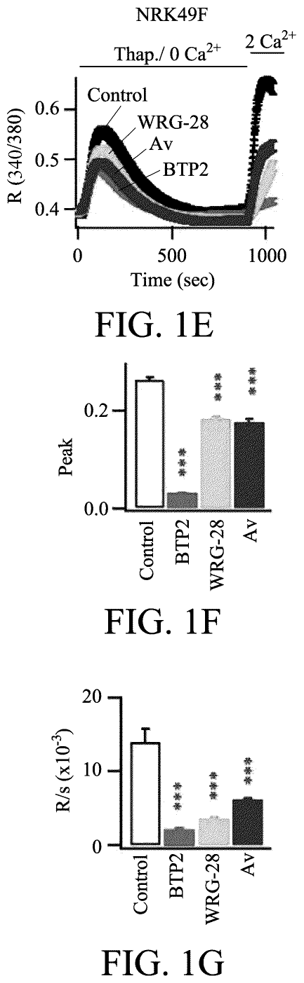

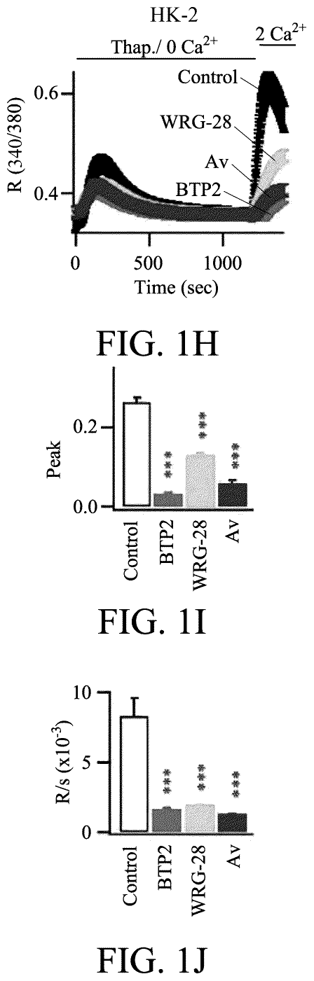

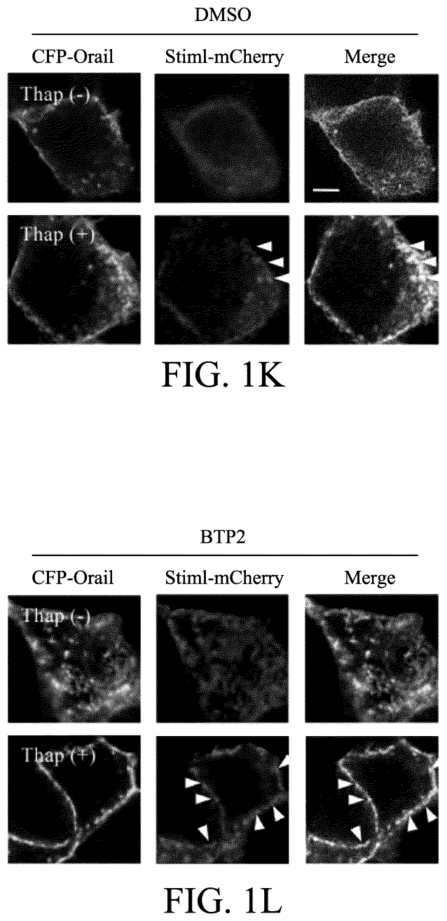

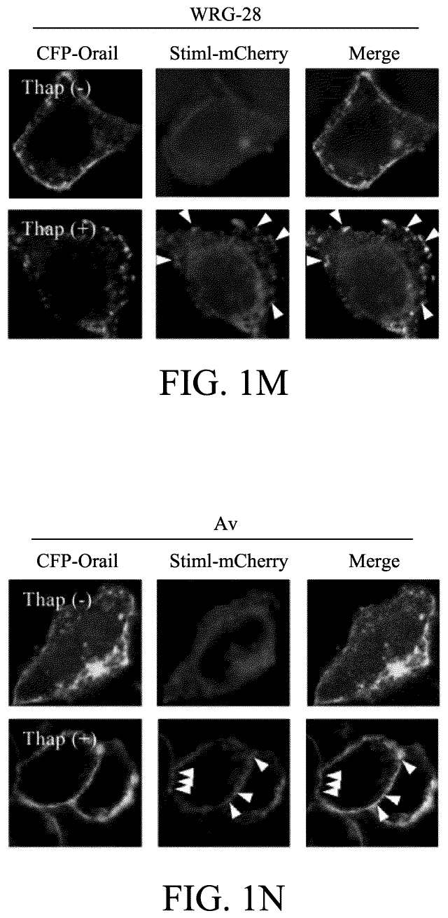

The results of 3D structure drug screening suggest that Av, Dh, and Th share structural similarities with WRG-28 and BTP2. To examine the impact of these compounds on DDR2 activation, HEK293T cells overexpressing DDR2 were pretreated with various compounds. Subsequently, fibrillar collagen was introduced into the medium and incubated for 12 hours. The activation of DDR2 was evaluated using an anti-phosphotyrosine antibody. WRG-28 has been utilized as a positive control. The activation of DDR2 was significantly reduced in cells pretreated with Av, Dh, and Th (FIG. 1A).

Next, we investigated the effects of WRG-28, Av, Dh, and Th on SOCE in HEK293T cells. WRG-28 and Av, both significantly reduced the second peak and rate of SOCE, whereas Dh and Th did not show significant effects (FIG. 1B, FIG. 1C, and FIG. 1D). Due to their structural similarity, WRG-28 and Av hold significant potential for targeting Orai1, the pore-forming domain of CRAC channels, which predominantly govern SOCE in HEK293T cells. The effects of WRG-28 and Av on SOCE were also tested in rat kidney fibroblast (NRK49F) cells (FIG. 1E, FIG. 1F, and FIG. 1G) and human proximal tubular cells (HK-2) (FIG. 1H, FIG. 1I, and FIG. 1J).

To verify the inhibitory effects of WRG-28 and Av on CRAC channel activation, HEK293T cells overexpressing CFP-Orai1 and Stim1-mCherry were pretreated with different compounds. Subsequently, CRAC channel activation was induced by applying thapsigargin, a sarcoendoplasmic reticulum calcium transport ATPase (SERCA) pump inhibitor. The formation of Stim1 or Orai1 puncta induced by store depletion was unaffected by pretreatment with BTP2, WRG-28, or Av (FIG. 1K, FIG. 1L, FIG. 1M, and FIG. 1N). This suggests that the aggregation of Stim1 or the coupling between Stim1 and Orai1 is not affected by these compounds.

### Example 2. WRG-28 and Av Suppress TGF-β1-Induced Fibroblast Activation and the Differentiation of Pericytes into Myofibroblasts

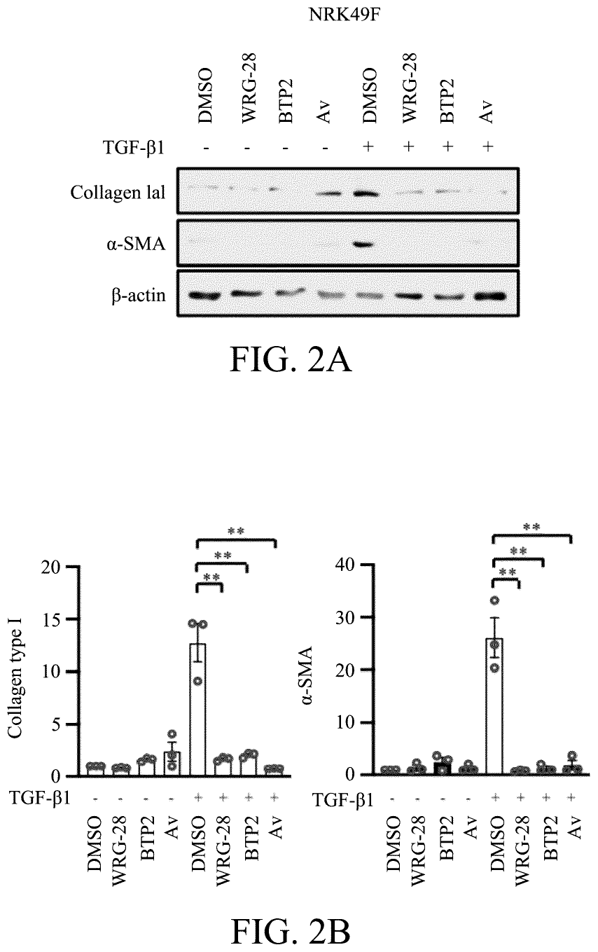

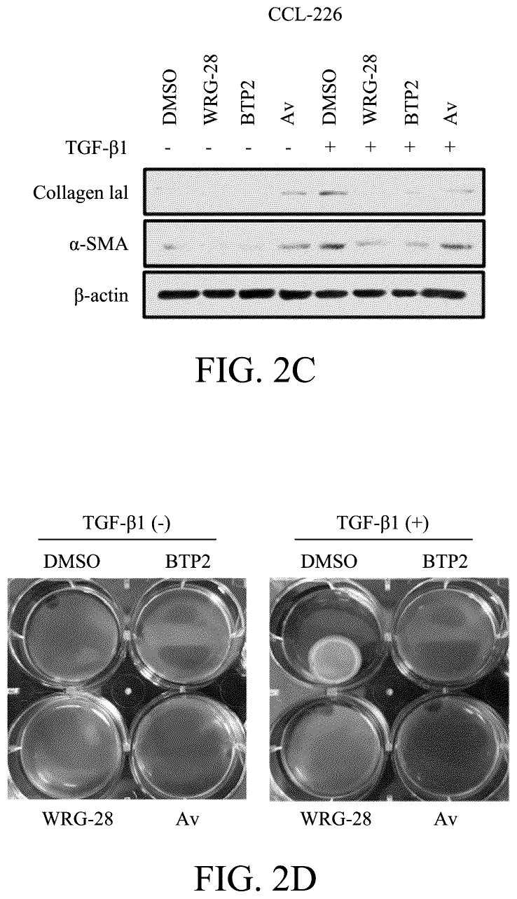

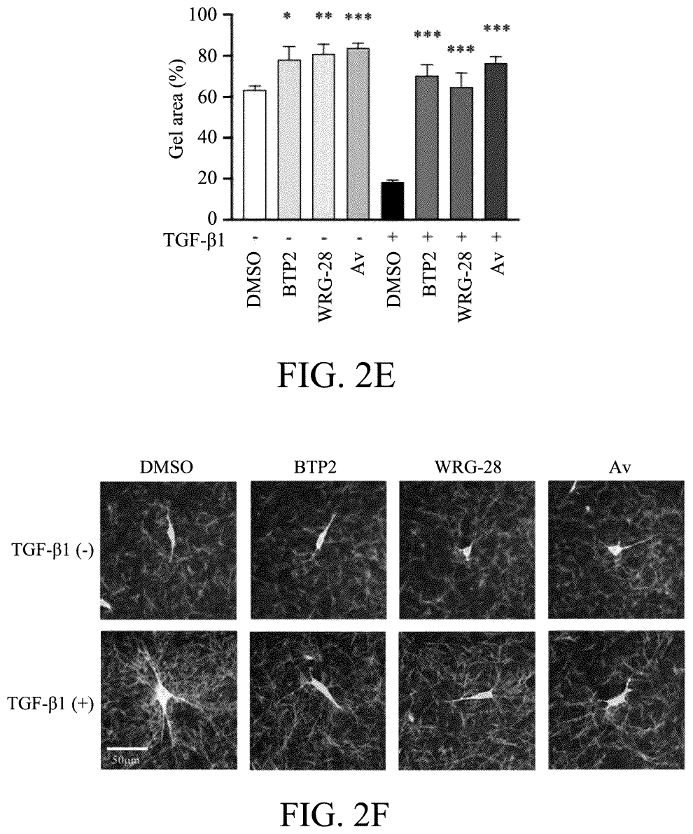

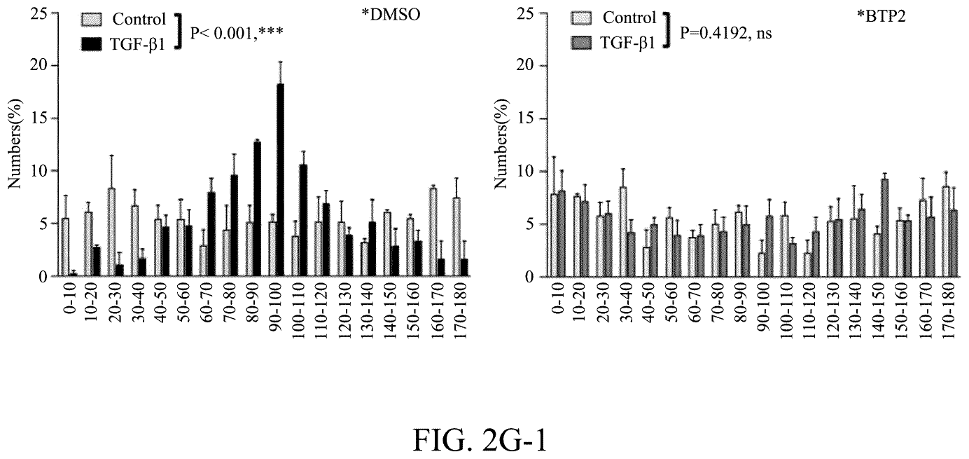

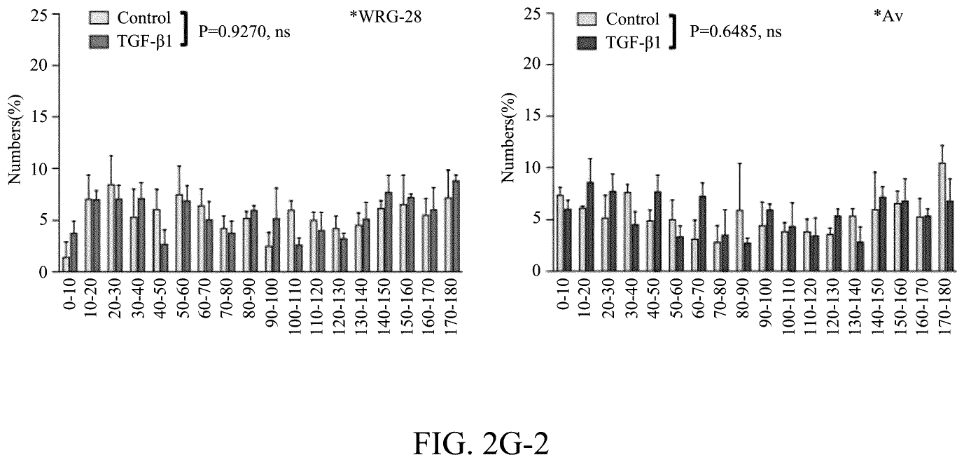

Both DDR2 and CRAC channels are involved in fibroblast activation. Consequently, we conducted tests to assess the effects of various compounds on rat renal fibroblasts. TGF-β1-induced upregulation of collagen 1a1 and α-SMA was significantly suppressed by pretreating NRK49F cells with BTP2, WRG-28, or Av (FIG. 2A and FIG. 2B). TGF-β1 treatment also induces the upregulation of collagen 1a1 and α-SMA in pericyte (CCL-226), which was significantly suppressed by pretreatment of BTP2, WRG-28, or Av (FIG. 2C). The results suggest the potent effects of WRG-28 and Av in inhibiting TGF-β1-induced fibroblast activation and pericyte-to-myofibroblast differentiation.

The alignment of the extracellular matrix (ECM) is a unique characteristic of myofibroblasts, altering the physical properties of the tissue microenvironment and posing a threat to tissue homeostasis during organ fibrosis. We then examined the effects of different compounds on the ECM alignment capability of myofibroblast following TGF-β1 stimulation. Representative figures showed that upon TGF-β1 stimulation, the gel area significantly decreased in the control group (DMSO) after release from culture dishes, suggesting an increase in contractile force in NRK49F cells. Furthermore, the gel shrinkage was markedly reduced in cells co-treated with BTP2, WRG-28, or Av (FIG. 2D and FIG. 2E). Upon examining the alignment of collagen fibers using FITC-labeled collagen, we observed perpendicular alignment of collagen fibers around the cells, particularly in the TGF-β1 treated group (FIG. 2F). Nevertheless, treatment with BTP2, WRG-28, or Av reduced the TGF-β1-induced collagen alignment (FIG. 2F, FIG. 2G-1, and FIG. 2G-2). Collectively, WRG-28 and Av inhibit TGF-β1-induced ECM remodeling.

### Example 3. WRG-28 and Av Inhibit the Activation of Cardiac, Pulmonary, and Hepatic Fibroblasts Induced by TGF-β1

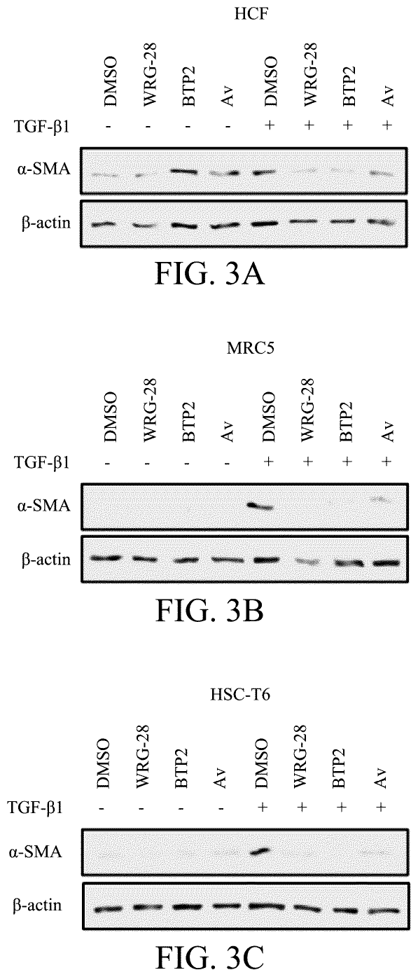

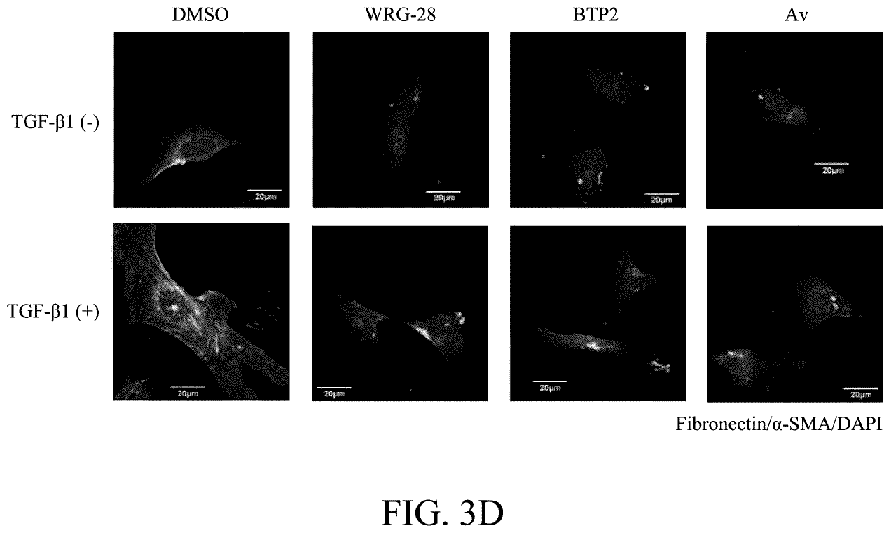

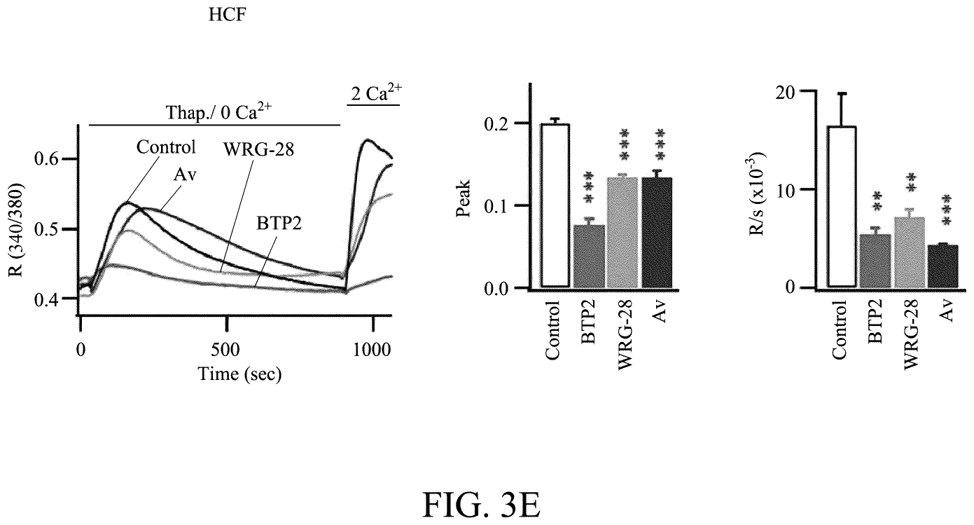

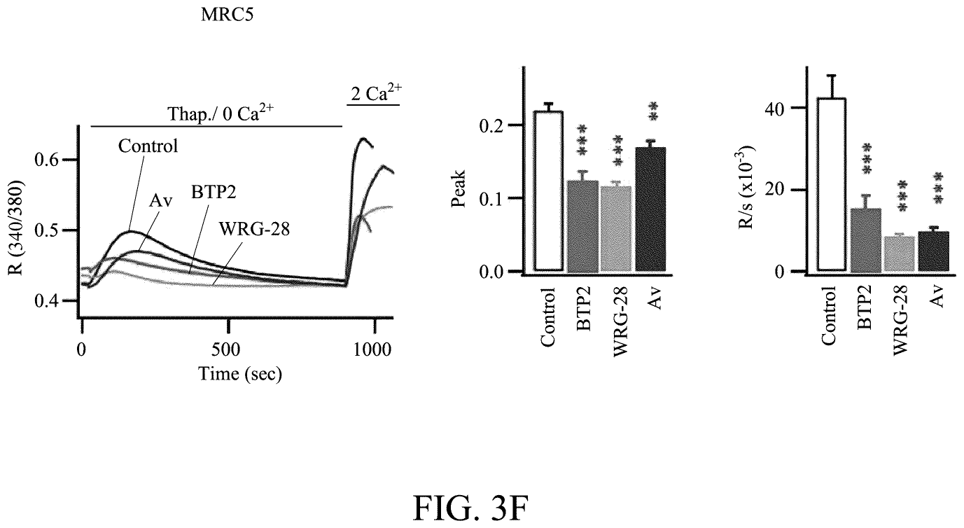

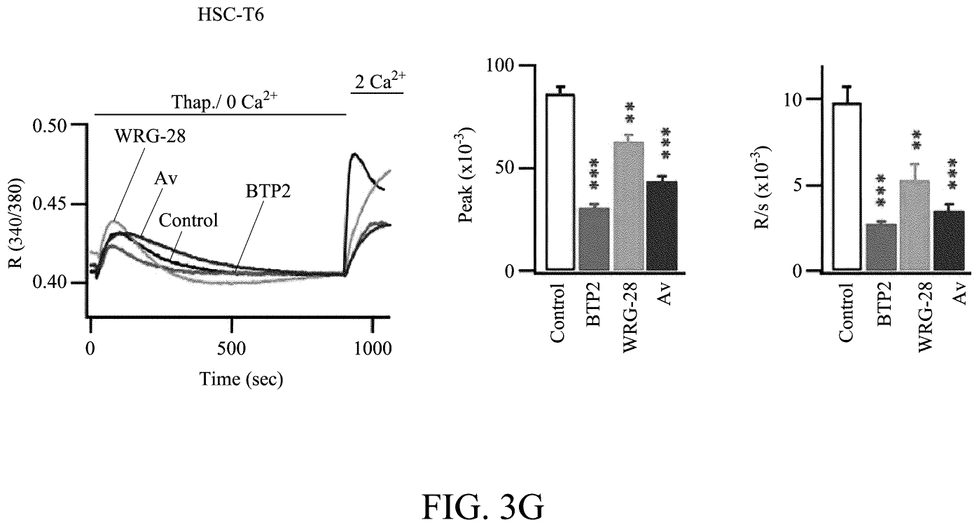

To assess the overall effects of WRG-28 and Av on TGF-1-induced fibroblast activation, human cardiac fibroblasts (HCF), human pulmonary fibroblasts (MRC5), and rat hepatic stellate cells (HSC-T6) were employed. The upregulation of α-SMA induced by TGF-β1 treatment was observed, but this effect was suppressed by the co-treatment with various compounds, including BTP2, WRG-28, and Av, across all tested cell (FIG. 3A, FIG. 3B, and FIG. 3C). The expression of fibronectin and α-SMA induced by TGF-β1 in HCF was examined, and once again, the treatment with BTP2, WRG-28, or Av suppressed this effect (FIG. 3D). Additionally, the treatment with TGF-β1 significantly increased the cell spreading area, a phenomenon that was mitigated by the co-treatment with BTP2, WRG-28, or Av in HCF (FIG. 3D).

Cytosolic calcium measurement was conducted to verify the effects of WRG-28 or Av on SOCE in various cell lines. In HCF (FIG. 3E), MRC5 (FIG. 3F), and HSC-T6 (FIG. 3G), the treatment with WRG-28 or Av notably reduced SOCE. These results suggested a general effect of different compounds in alleviating TGF-β1-induced myofibroblast activation.

### Example 4. WRG-28 and Av Reduce the Secretion of Cytokines in T Cells and Macrophages

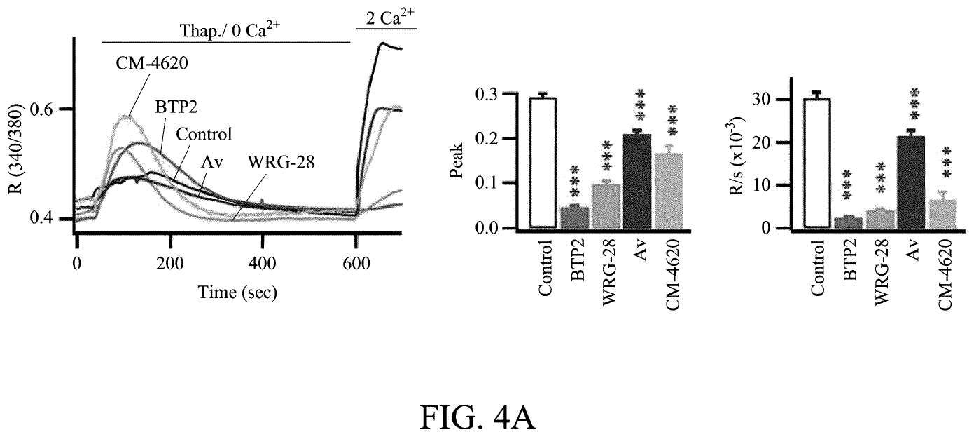

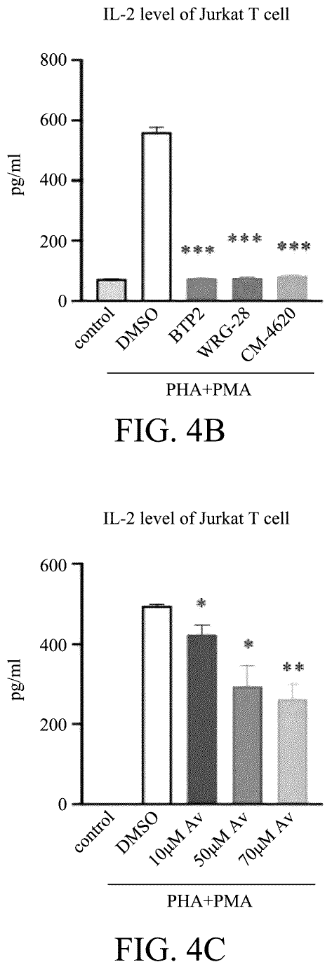

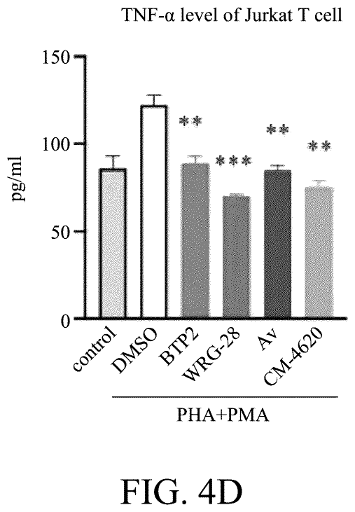

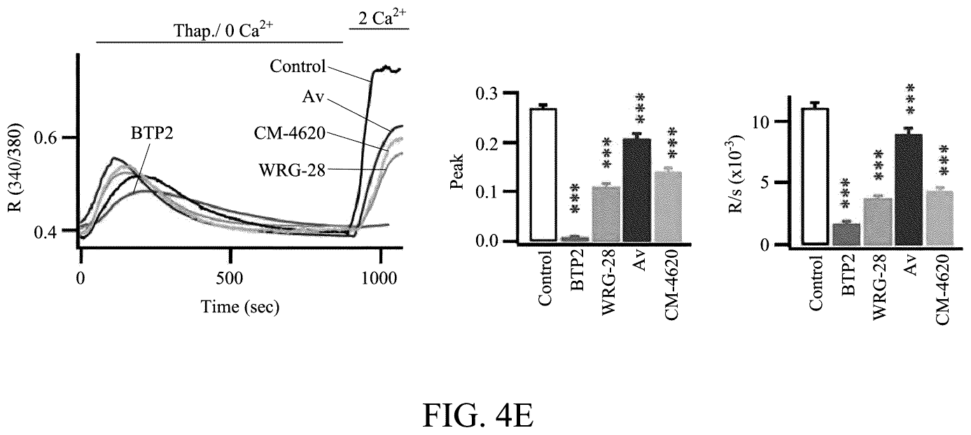

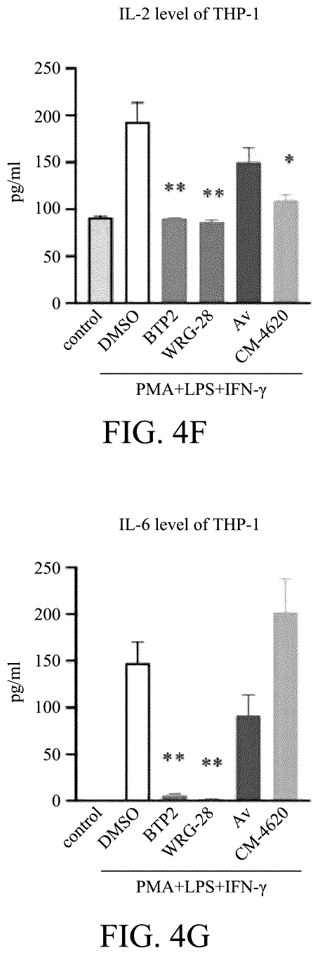

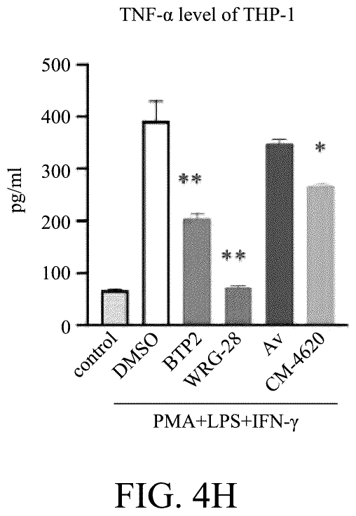

CRAC channel activation is involved in T-cell differentiation and cytokine expression. Subsequently, we tested the effects of WRG-28 and Av on SOCE and cytokine secretion in both T cells and macrophages. The treatment with WRG-28 and Av demonstrates varying capabilities in blocking SOCE in T cells (FIG. 4A) and macrophages (FIG. 4E). Overall, WRG-28 exhibits higher efficacy compared to Av and demonstrates a similar potential to CM-4620, the newest FDA-approved compound used to block cytokine release, in inhibiting SOCE. Av exhibits lower potency but still significantly reduces SOCE in both T cells (FIG. 4A) and macrophages (FIG. 4E). The efficacy in blocking SOCE is reflected in cytokine release. WRG-28, along with BTP2 and CM-4620, significantly reduced the secretion of IL-2 (FIG. 4B) and TNF-β (FIG. 4D) in T cells, as well as IL-2 (FIG. 4F), IL-6 (FIG. 4G), and TNF-β (FIG. 4H) in macrophages. Conversely, Av demonstrates relatively lower potential but still significantly reduces IL-2 (FIG. 4C) and TNF-β (FIG. 4D) secretion in T cells. It exhibits a minor reduction in the secretion of IL-2 (FIG. 4F), IL-6 (FIG. 4G), and TNF-β (FIG. 4H) in macrophages. These results suggest that the varying capabilities of WRG-28 and Av in SOCE can modulate the immune response to different extents.

### Example 5. WRG-28 and Av Reduce UUO-Induced Renal Fibrosis

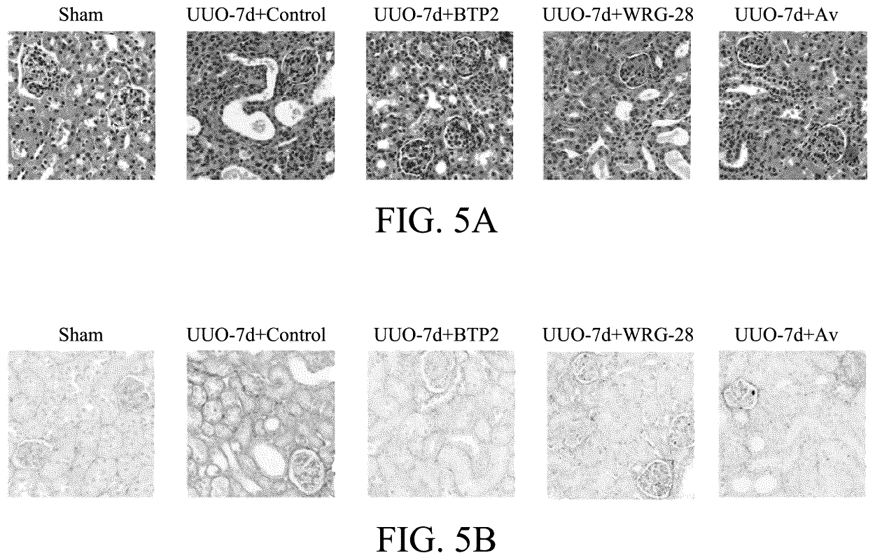

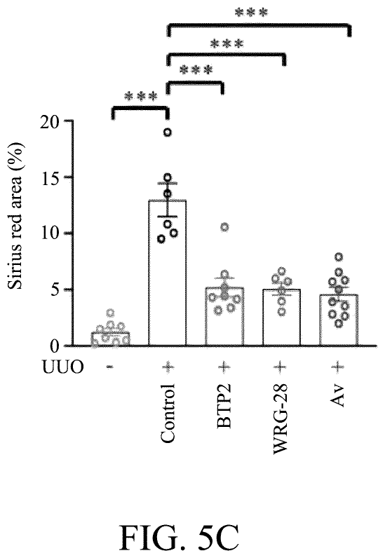

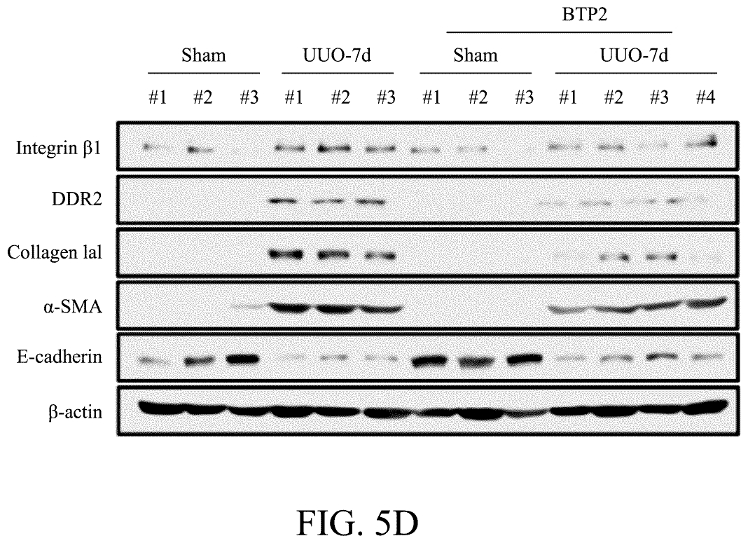

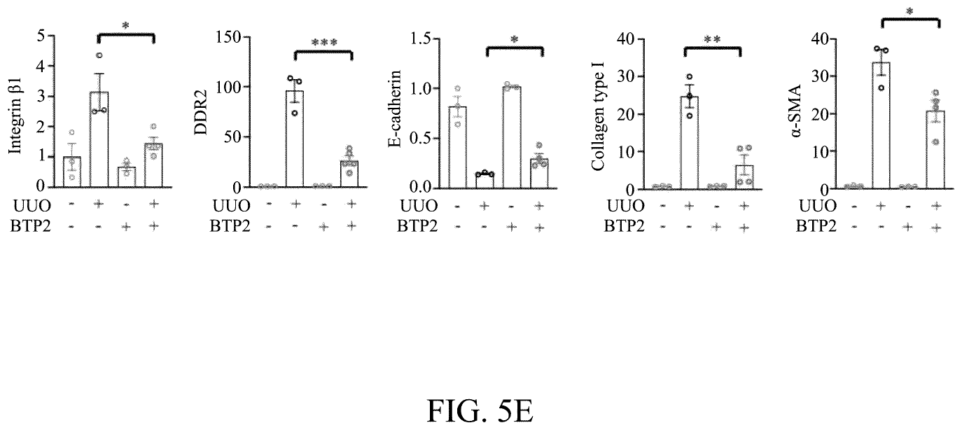

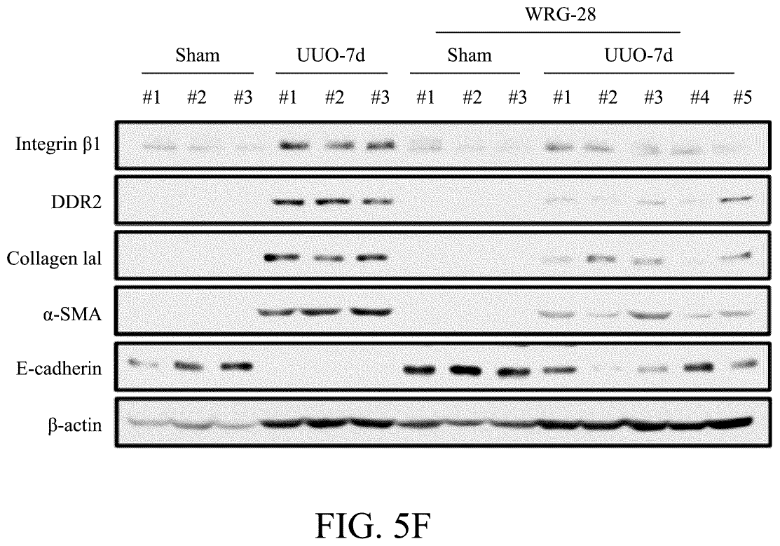

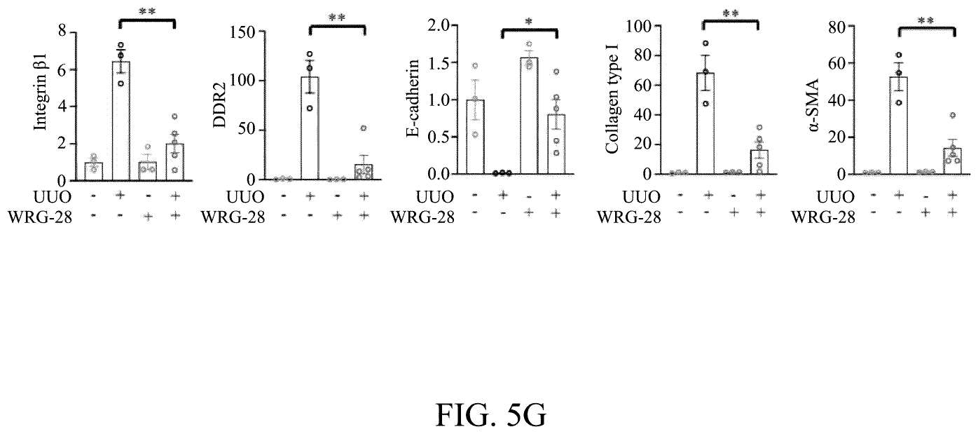

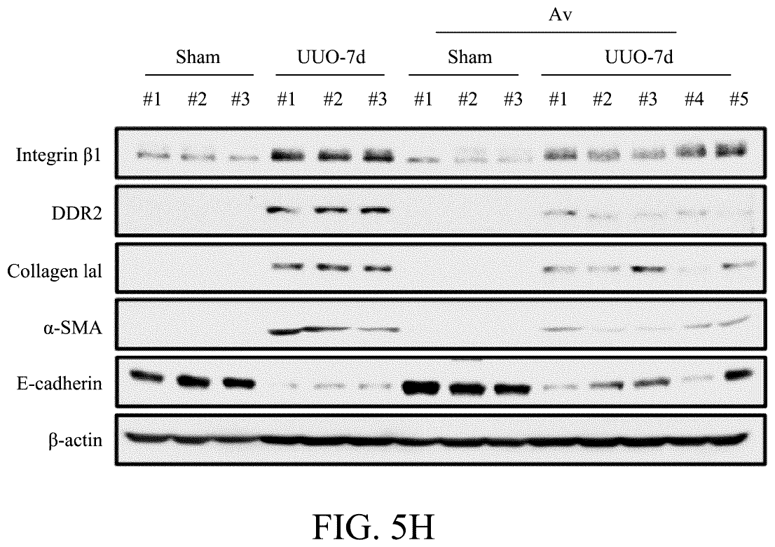

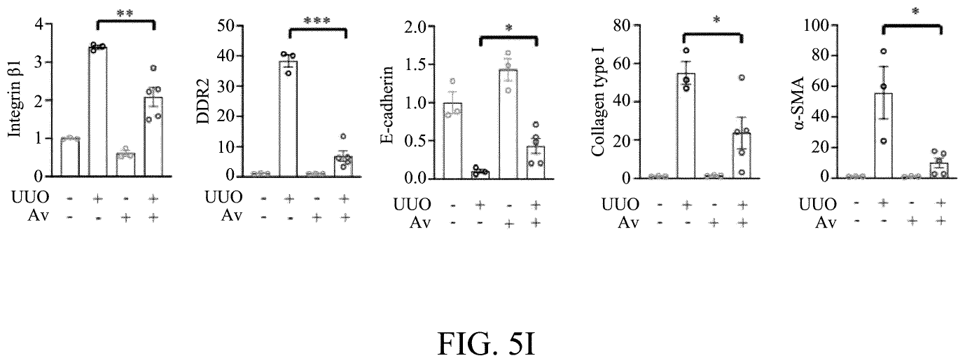

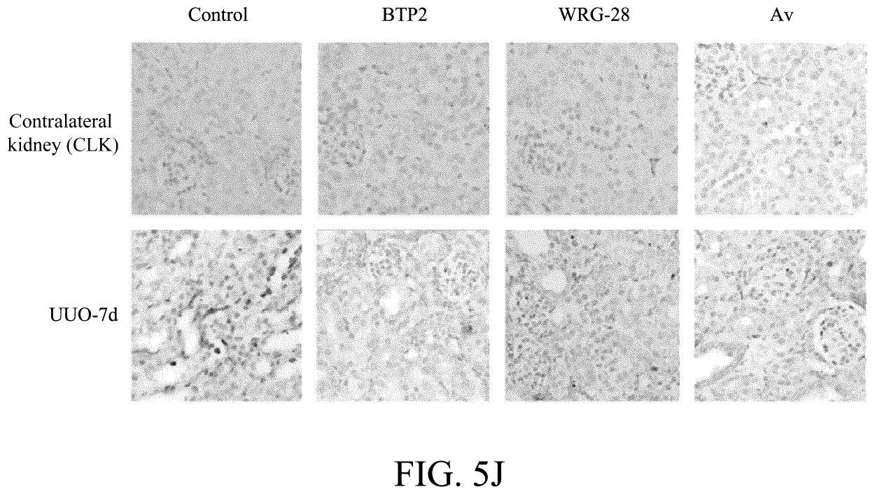

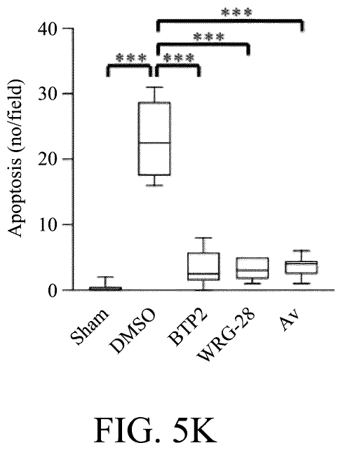

To evaluate the efficacy of WRG-28 and Av in protecting the kidney from fibrotic injury, we conducted the conventional unilateral ureteral obstruction (UUO) surgery. Mice underwent the UUO surgery and were simultaneously administered the studied compounds for 7 days. The histology results showed that the UUO treatment induced tubular dilation and interstitial expansion. Nevertheless, the administration of BTP2, WRG-28, or Av appeared to mitigate the effects caused by UUO (FIG. 5A). The results from Sirius red staining revealed an accumulation of fibrillar collagen in tubulointerstitial space after 7 days of UUO surgery. However, the administration of BTP2, WRG-28, or Av significantly reduced the area positively stained with fibrillar collagen (FIG. 5B and FIG. 5C).

The protein analysis results revealed an upregulation of mesenchymal marker proteins, such as integrin β1, DDR2, collagen 1a1, and α-SMA, along with a downregulation of the epithelial marker protein, E-cadherin, in kidneys subjected to UUO treatment. Administration of BTP2 (FIG. 5D and FIG. 5E), WRG-28 (FIG. 5F and FIG. 5G), or Av (FIG. 5H and FIG. 5I) suppressed the UUO-induced upregulation of integrin β1, DDR2, collagen 1a1, and α-SMA, while also reversing the UUO-induced downregulation of E-cadherin. These findings suggest the potency of WRG-28 and Av in inhibiting tubulointerstitial fibrosis.

The loss of tubular epithelial cells leads to tubular atrophy and impairs kidney functions. In UUO-treated kidneys, there was a significant increase in apoptotic tubular cells, which was notably reduced in mice treated with BTP2, WRG-28, or Av (FIG. 5J and FIG. 5K). These results suggest the protective effects of BTP2, WRG-28, and Av against UUO-induced tubular atrophy and apoptosis.

### Example 6. WRG-28 and Av Protect and Promote Tissue Repair from Obstruction-Induced Tissue Injury and Fibrosis

The gradual increase in hydrostatic pressure and toxicity from accumulated excretes leads to irreversible injury in conventional UUO. Another disadvantage of conventional UUO is the inability to assess the remaining kidney function on the injured side. Therefore, to gain further insight into the effects of the studied compounds on kidney protection or repair after injury, we conducted the reversal unilateral ureteral obstruction (RUUO) surgery. The left ureter was surgically ligated for 7 days, after which it was re-connected to the bladder. Our recent results indicate that the injury and fibrotic markers are reversible within 3 days of ligation followed by surgical re-connection. However, when ligation persisted for 7 days followed by re-connection, it led to irreversible and persistent progression of fibrosis.

To assess whether the administration of WRG-28 or atovaquone (Av) promotes kidney repair, mice were injected with WRG-28 or atovaquone (Av) for 7 days during ureter obstruction (7*/15d). After the obstruction was released for 13 days, the right ureter was ligated to enable the assessment of kidney function by measuring the BUN and CRE levels in the blood.

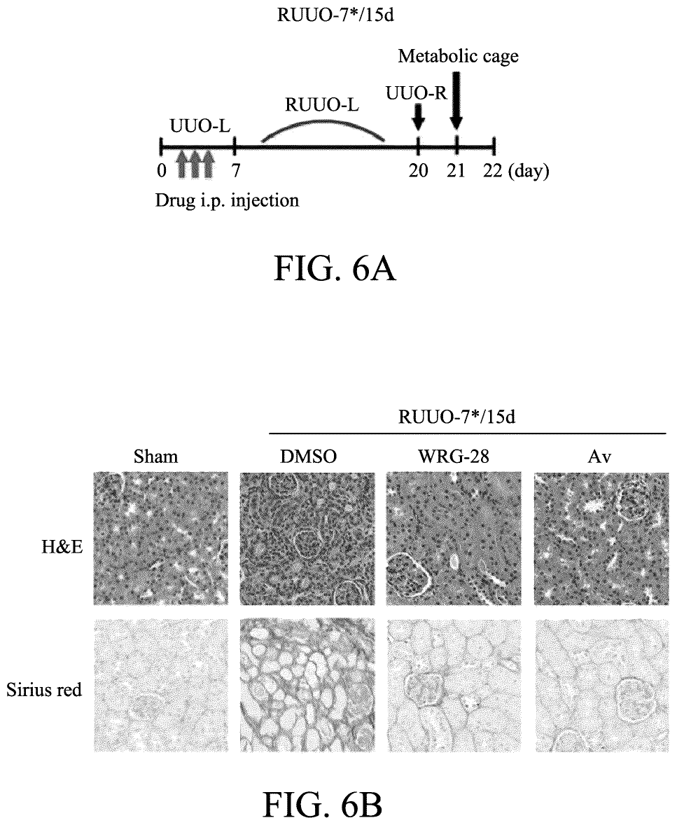

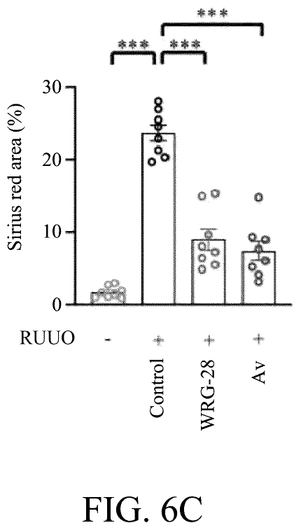

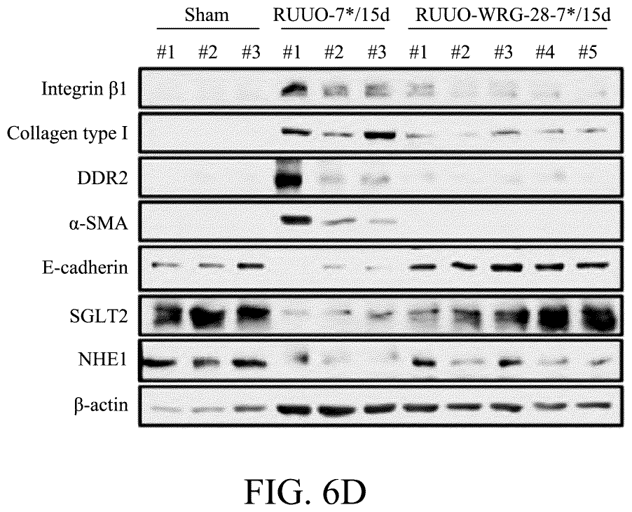

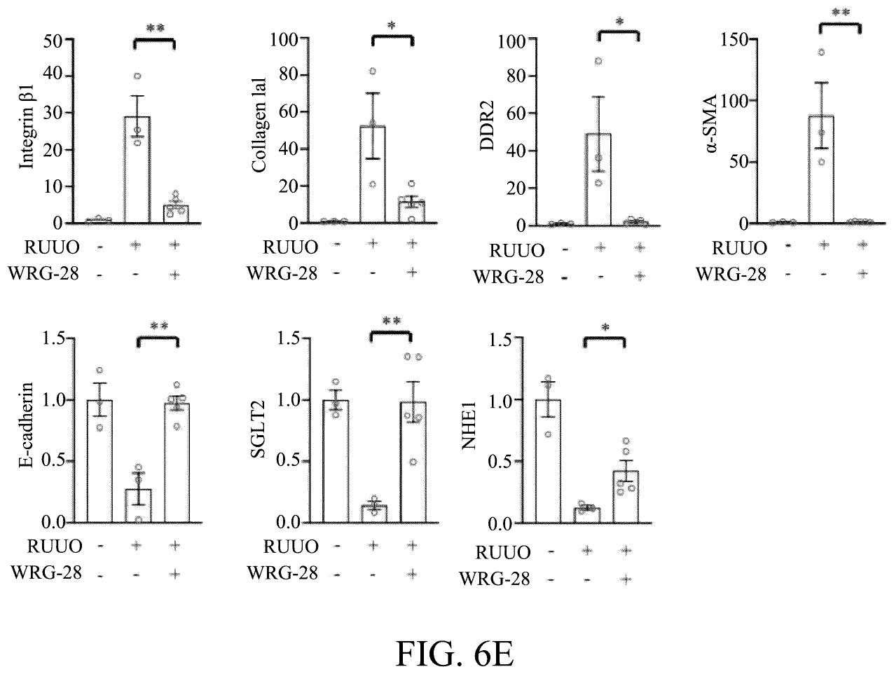

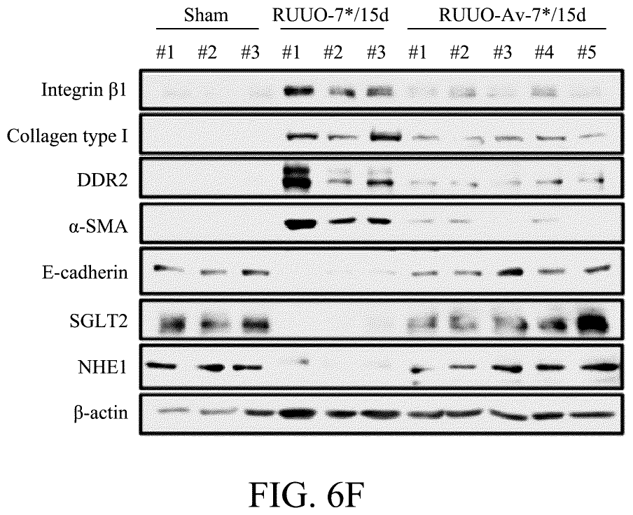

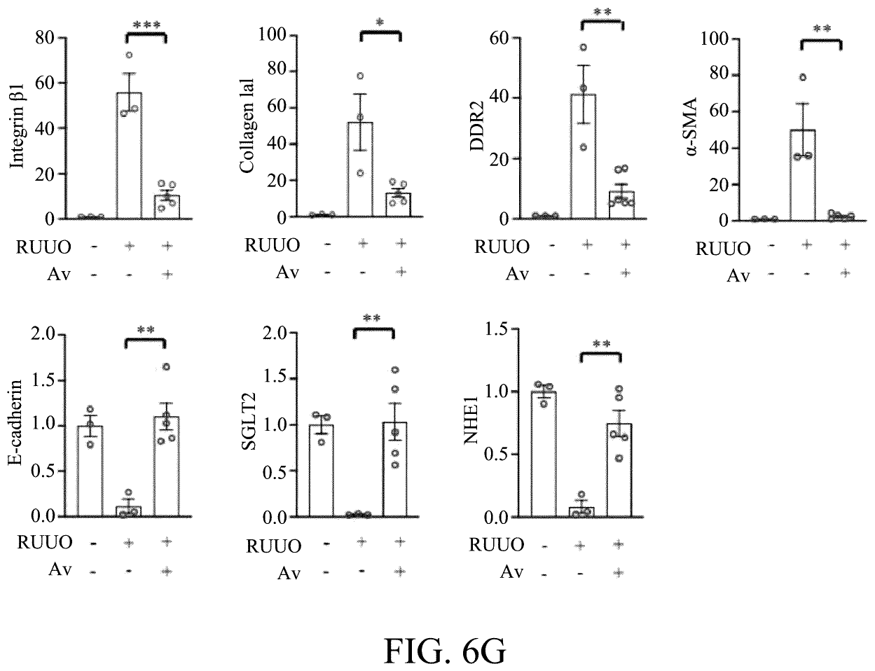

The experimental flow was illustrated in FIG. 6A. The histology results revealed severe fibrotic scarring in the DMSO treated group; however, the administration of WRG-28 and Av preserved the intact tubular structure (FIG. 6B). The collagen-positive area increased from 2% to 22% (FIG. 6C) after the obstruction was released in the control group, but the treatment with WRG-28 or Av significantly reduced the expansion of the fibrotic area (FIG. 6B and FIG. 6C).

An obstruction for 7 days followed by release for 13 days led to irreversible fibrosis and sustained high levels of mesenchymal marker proteins, including integrin β1, DDR2, collagen 1a1, and α-SMA, along with low levels of epithelial marker proteins, such as E-cadherin, SGLT2 and NHE1. The administration of WRG-28 (FIG. 6D and FIG. 6E) and Av (FIG. 6F and FIG. 6G) during obstruction significantly reduced the expression of mesenchymal marker proteins. Importantly, there was a significant increase in the expression of epithelial markers in mice treated with WRG-28 (FIG. 6D and FIG. 6E) and Av (FIG. 6F and FIG. 6G).

### Example 7. WRG-28 or Av Treatment Reduces Obstruction-Induced Persistent Progression of Renal Fibrosis after the Obstruction is Released

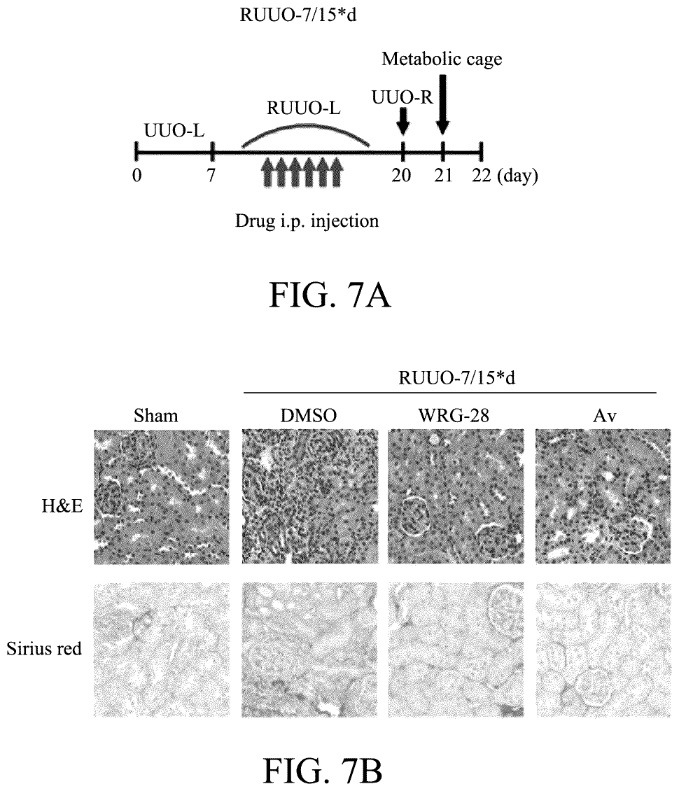

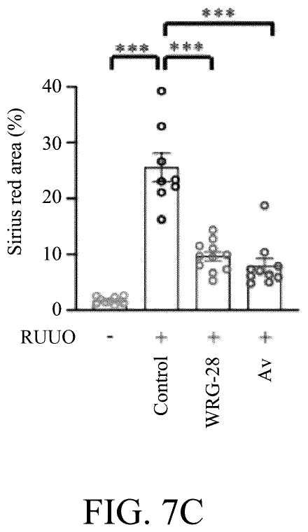

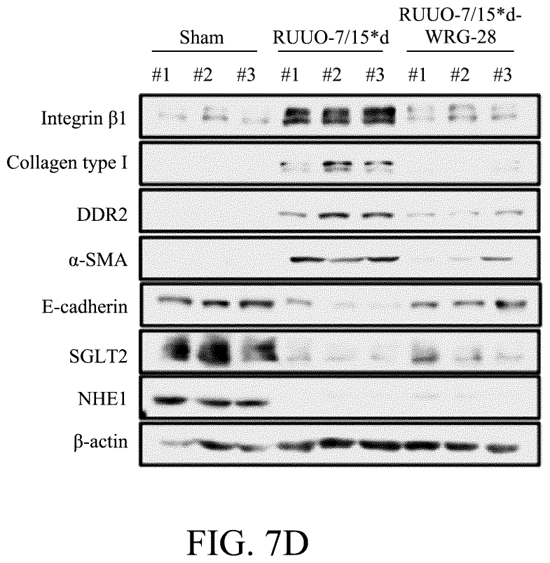

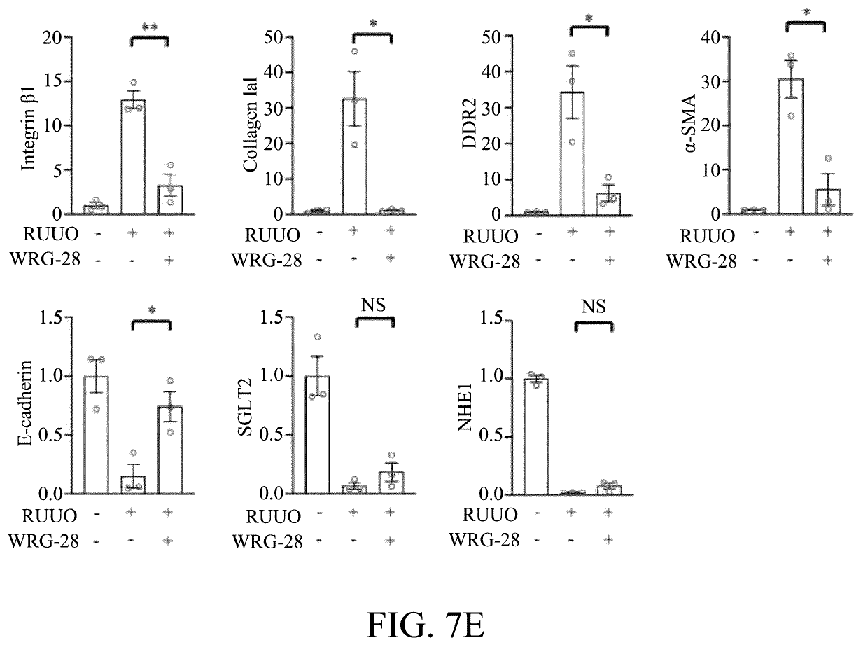

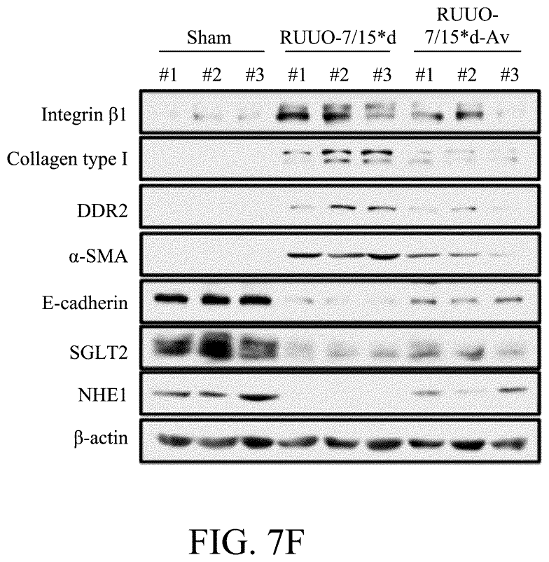

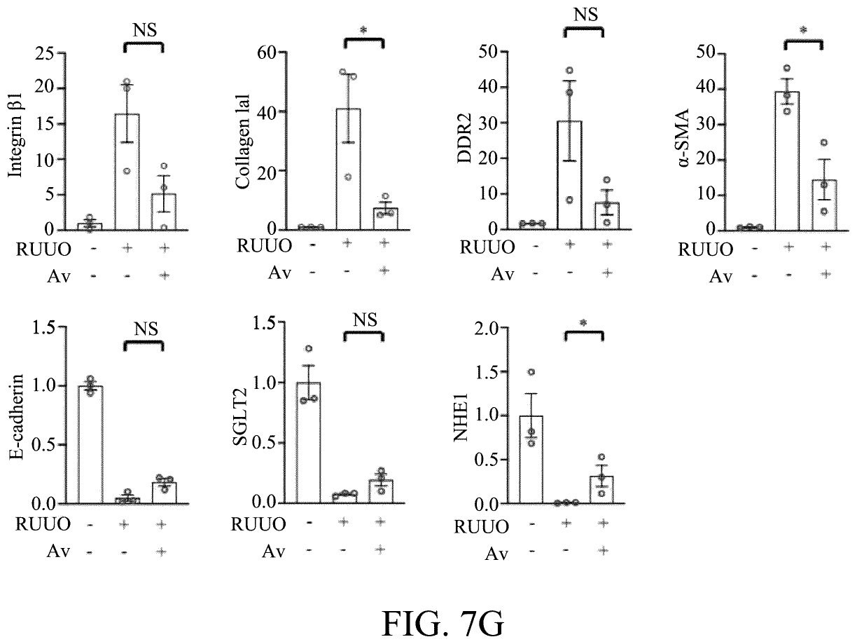

Next, we tested whether the administration of WRG-28 or Av could promote kidney repair and reduce the persistent progression of fibrosis after the obstruction was released. Mice received injections of WRG-28 or Av for the following 13 days after the release of ureter obstruction (7/15*d) (FIG. 7A). The WRG-28 and atovaquone (Av) treated groups exhibited numerous intact tubular structures, less inflammation and scar tissue compared to the DMSO-treated mice (FIG. 7B, upper panel). The collagen-positive area was also reduced due to WRG-28 or Av treatment (FIG. 7B, lower panel, and FIG. 7C). Obstruction-induced upregulation of integrin β1, DDR2, collagen 1a1, and α-SMA was alleviated by WRG-28 (FIG. 7D and FIG. 7E) and Av (FIG. 7F and FIG. 7G) treatment. However, the downregulation of epithelial marker proteins, E-cadherin, SGLT2 and NHE1, was only partially reversed in the WRG-28 (FIG. 7D and FIG. 7E) and Av (FIG. 7F and FIG. 7G) treated groups. These results suggest that the administration of WRG-28 and Av can reverse fibrosis and promote tissue repair.

### Example 8. WRG-28 and Av Restore Epithelial Differentiation and Reduce Collagen Deposition and Myofibroblast Expansion in the Obstruction-Induced Persistent Progression of Fibrosis

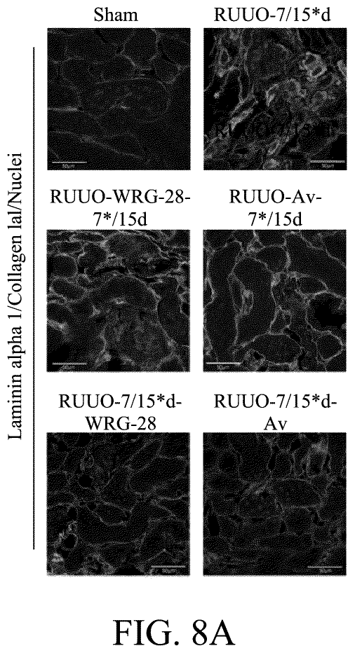

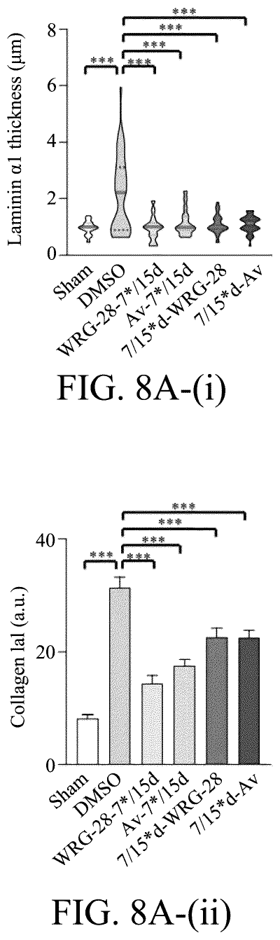

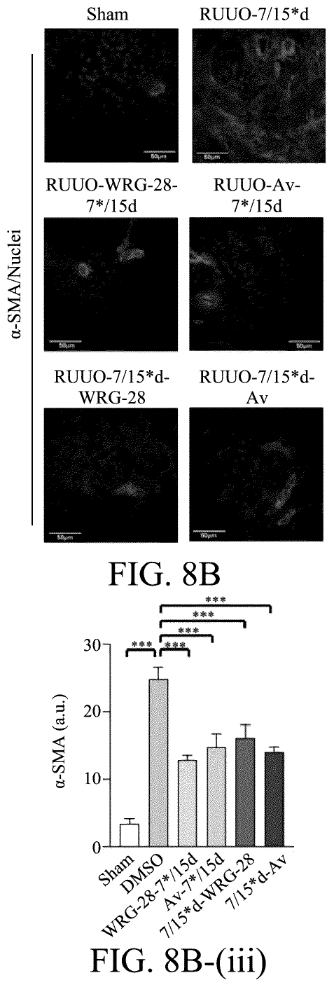

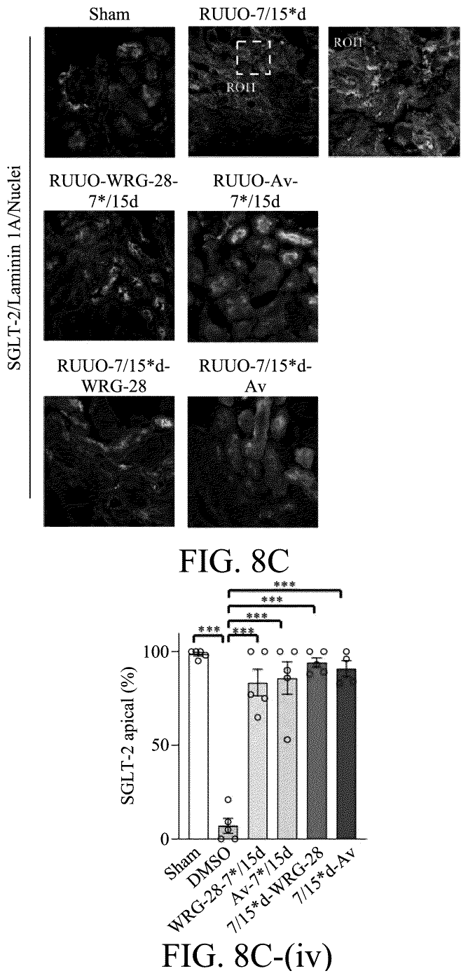

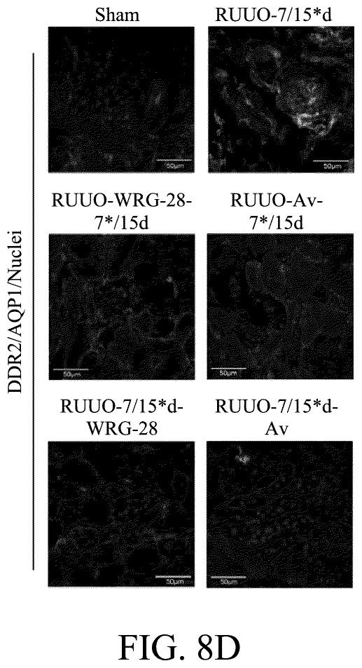

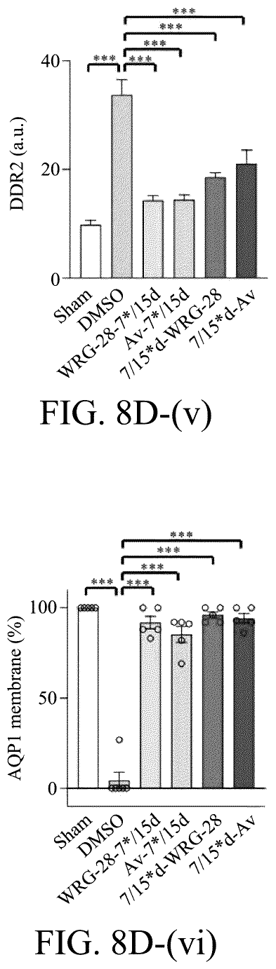

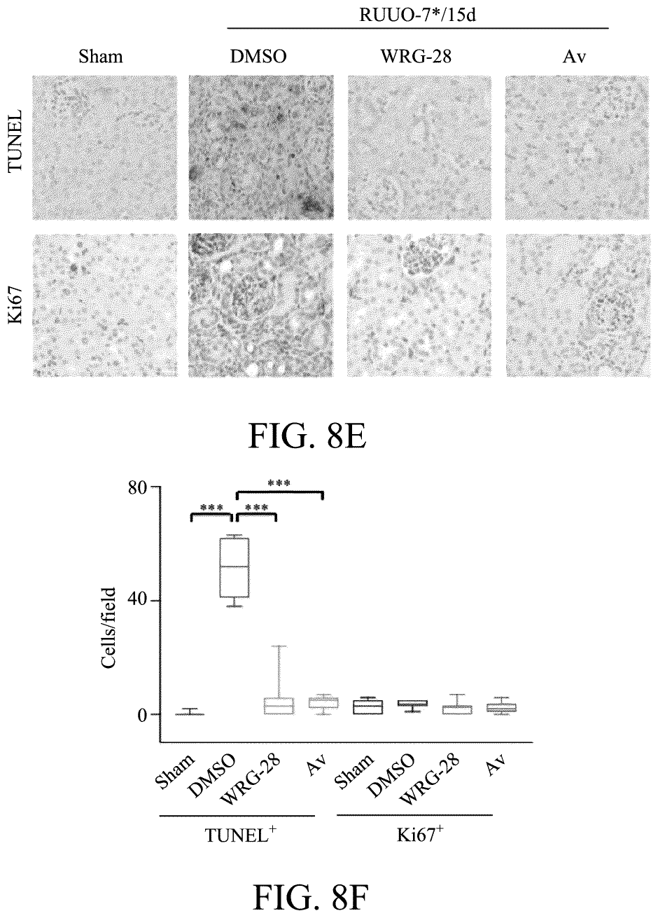

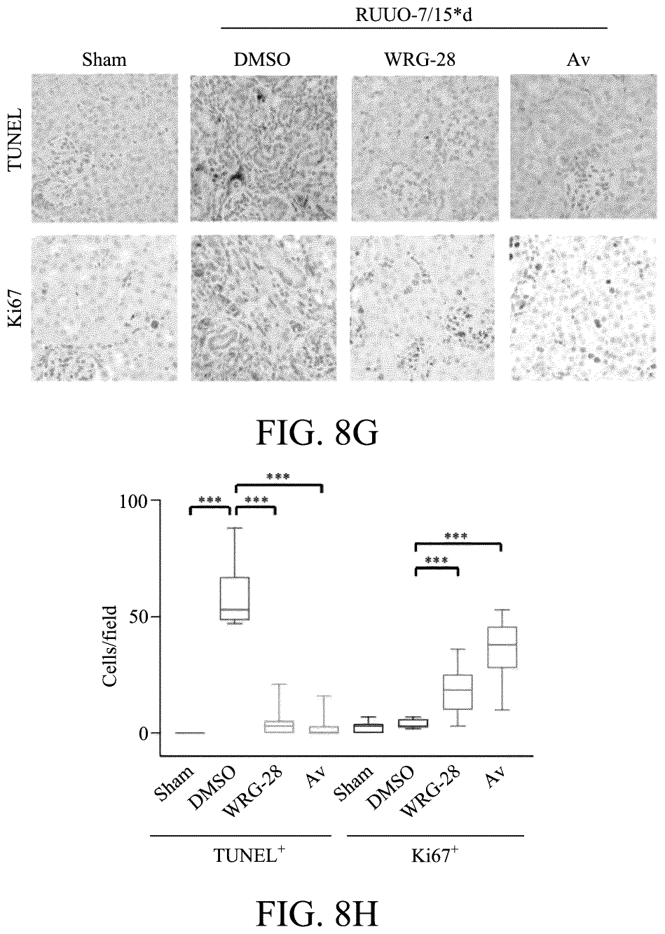

Immunofluorescence studies further examined the alignment and expression features of ECMs in kidneys treated with different compounds at various time points. Laminin alpha 1, the primary component of the basement membrane, forms thin layers beneath the tubular epithelial cells and between the podocyte and endothelial cells in Bowman's capsule in the control kidney (sham; shown in FIG. 8A). Tubular atrophy accompanied by thickening of the basement membrane was prominently observed in the kidney subjected to obstruction for 7 days and then released for 15 days (RUUO-7/15*d-DMSO) (FIG. 8A and FIG. 8A-(i)). The average thickness was significantly increased compared to the sham group (FIG. 8A-(i)). However, in the WRG-28 and atovaquone (Av) treated-groups, whether during the obstruction (7*/15d) or after its release (7/15*d), they consistently exhibited a uniform and unchanging width of the basement membrane (FIG. 8A and FIG. 8A-(i)). Most notably, fewer atrophic tubules were observed in the groups treated with WRG-28 and Av (FIG. 8A and FIG. 8A-(i)). Also, the expression of collagen 1a1 was markedly elevated in the DMSO treated group (RUUO-7/15*d-DMSO), while the administration of WRG-28 and Av mitigated the accumulation of collagen 1a1 (FIG. 8A and FIG. 8A-(ii)). The expression of α-SMA was significantly elevated in the DMSO treated group, whereas it was markedly reduced in the WRG-28 and Av treated groups (FIG. 8B and FIG. 8B-(iii)). The collagen upstream signal, DDR2, was significantly upregulated in the DMSO-treated group (RUUO-7/15*d-DMSO) (FIG. 8D and FIG. 8D-(v)); however, the administration of WRG-28 and Av reduced the expression of DDR2 compared to the DMSO-treated group (FIG. 8D and FIG. 8D-(v)). These results suggest that the administration of WRG-28 and Av reduces myofibroblast activation and the subsequent reassembly of ECMs during renal fibrosis.

The integrity and structure of the basement membrane influence epithelium differentiation, and the arrangement of specialized membrane microdomains is one of its defining characteristics. Sodium-glucose cotransporter-2 (SGLT-2) is specifically expressed in proximal tubular cells and is localized in the apical lumen in the healthy kidney (sham, FIG. 8C and FIG. 8C-(iv)). Obstruction for 7 days followed by a release for 15 days (RUUO-7/15*d-DMSO) induced downregulation of SGLT-2 (FIG. 7D, FIG. 7F, FIG. 8C, and FIG. 8C-(iv)). Additionally, in cells expressing SGLT-2, the protein lacked apical membrane distribution in the kidneys of mice treated with DMSO only (FIG. 8C and FIG. 8C-(iv)). However, both WRG-28 and Av treatments reversed the apical membrane expression of SGLT-2. These phenomena were also evident in mice that received the treatment during obstruction followed by release, showing a distribution quite close to normal (FIG. 8C and FIG. 8C-(iv)), although the SGLT2 protein levels were only slightly altered (FIG. 7D, FIG. 7E, FIG. 7F, and FIG. 7G). Similar phenomena were also observed in other epithelial marker proteins, including AQP1 (FIG. 8D and FIG. 8D-(vi)) and NHE1 (data not shown). Taken together, administration of WRG-28 and Av enhanced tubular differentiation during renal fibrosis.

An extreme imbalance between apoptotic and proliferating tubular cells, partly due to cell senescence, leads to maladaptive repair in fibrotic kidneys. Apoptotic cells were significantly reduced by the administration of WRG-28 or Av during ureter obstruction (FIG. 5J and FIG. 5K). Even after the obstruction was released for 15 days, there were still numerous apoptotic (TUNEL-positive cells) tubular cells in DMSO-treated mice (FIG. 8E and FIG. 8F). However, only a few apoptotic cells were observed in the groups treated with WRG-28 and Av during obstruction, and the number remained low even after the obstruction was released for 15 days (FIG. 8E and FIG. 8F). The numbers of proliferating tubular cells (Ki67-positive) did not significantly differ among groups, but there were more proliferating interstitial cells in DMSO treated mice compared to WRG-28 and atovaquone (Av) treated groups (FIG. 8E and FIG. 8F). Administration of WRG-28 and Av after the obstruction was released also significantly reduced the number of apoptotic cells (TUNEL-positive cells) (FIG. 8G and FIG. 8H), suggesting both preventive and treatment effects contributed by them. Importantly, the administration of WRG-28 and Av after the obstruction was released also increased the numbers of proliferating tubular cells (Ki67-positive cells) (FIG. 8G and FIG. 8H). Therefore, based on the results above, WRG-28 and Av treatments could mitigate maladaptive repair and assist tubular regeneration.

### Example 9. Treatment with WRG-28 or Atovaquone (Av) Improves Kidney Function in Obstruction-Induced Kidney Injury

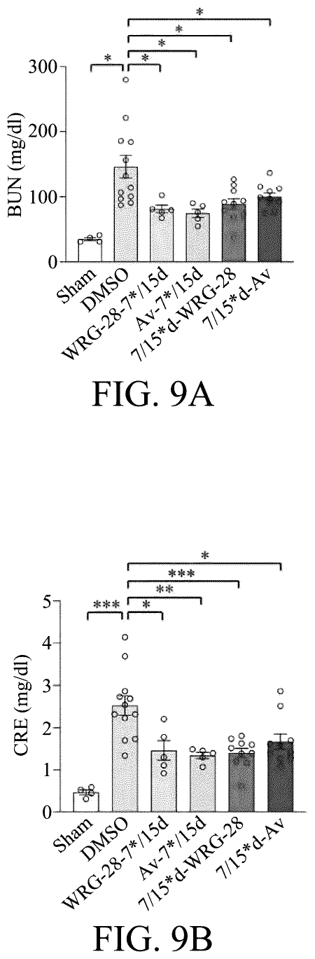

The levels of BUN and CRE in the serum were measured to evaluate kidney function. Obstruction-induced kidney injury and persistent fibrosis progression resulted in 2-3 times higher plasma BUN and CRE levels. However, administration of WRG-28 or Av significantly reduced the levels of BUN and CRE compared to those in DMSO-treated mice (FIG. 9A and FIG. 9B). These results indicate the effectiveness of WRG-28 and Av in treating and protecting kidney function.

### Example 10. Transcriptome Analysis in WRG-28 and Av Treated Mice

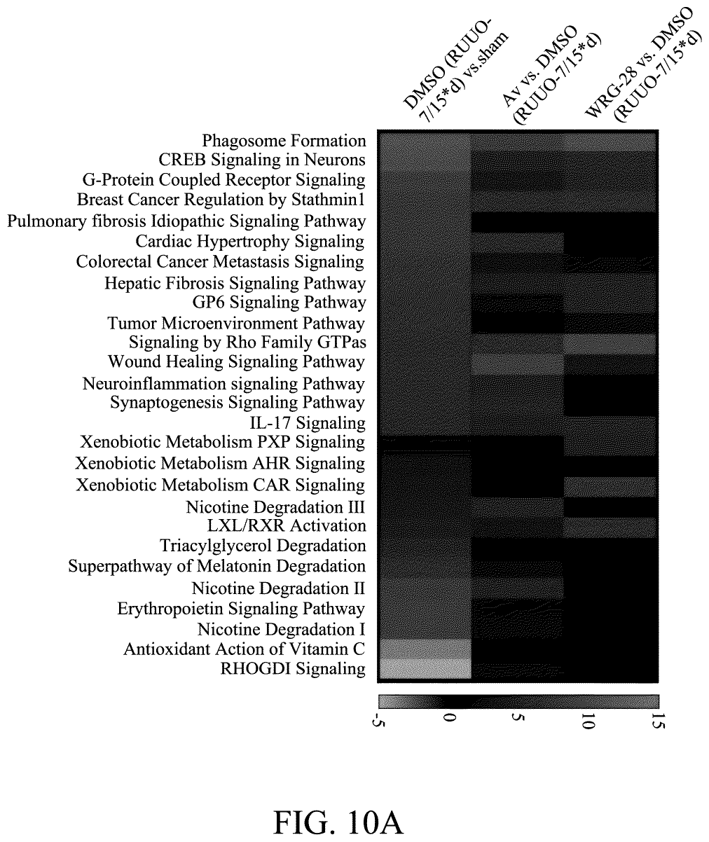

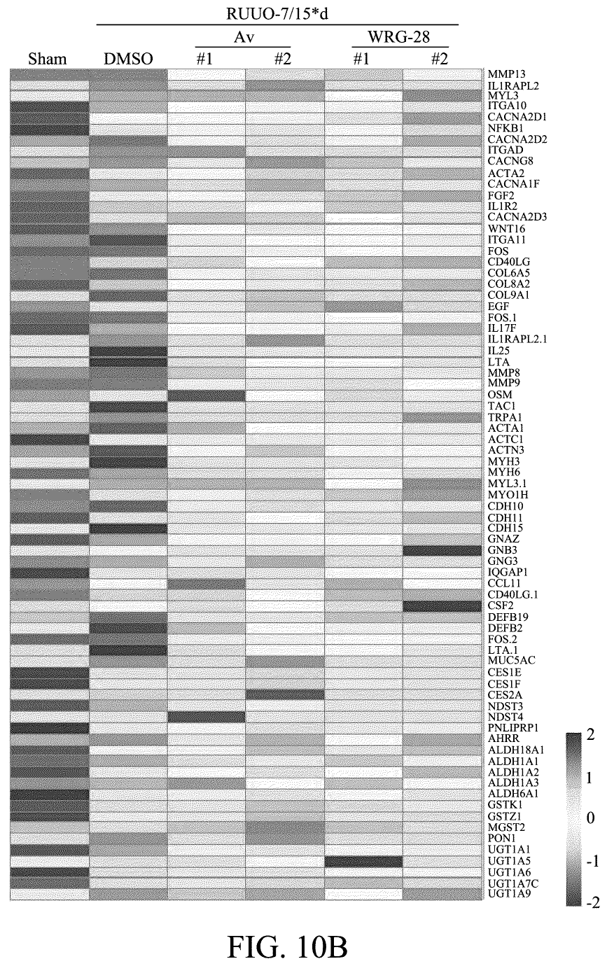

Total ribonucleic acid (RNA) was extracted from 2 groups: control mice (sham) and mice that received treatments with DMSO, WRG-28, or Av after the obstruction was released (7/15*d). Transcriptomes were then analyzed by ingenuity pathway analysis (IPA). The results indicated a significant increase in the inflammatory response, signaling, and fibrotic gene cohort in the DMSO-treated group compared to mice without surgical treatment. Conversely, the administrations of WRG-28 and Av reduced the inflammatory responses, and fibrotic signaling pathways (FIG. 10A). Some of those genes were listed in FIG. 10B. The large-scale transcriptome analysis further confirms the potent effects of WRG-28 and Av in preventing and protecting kidneys from progressive fibrosis.

### Example 11. Treatment with WRG-28 or Av Effectively Reduces Pulmonary Fibrosis

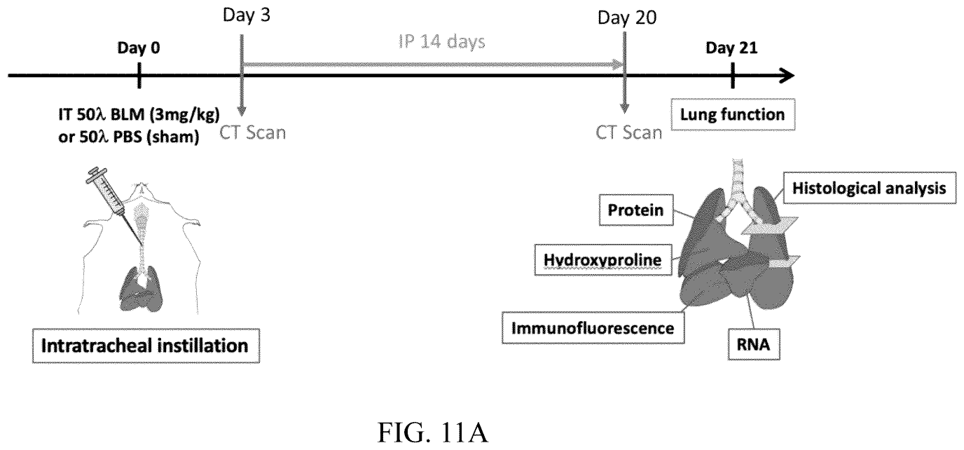

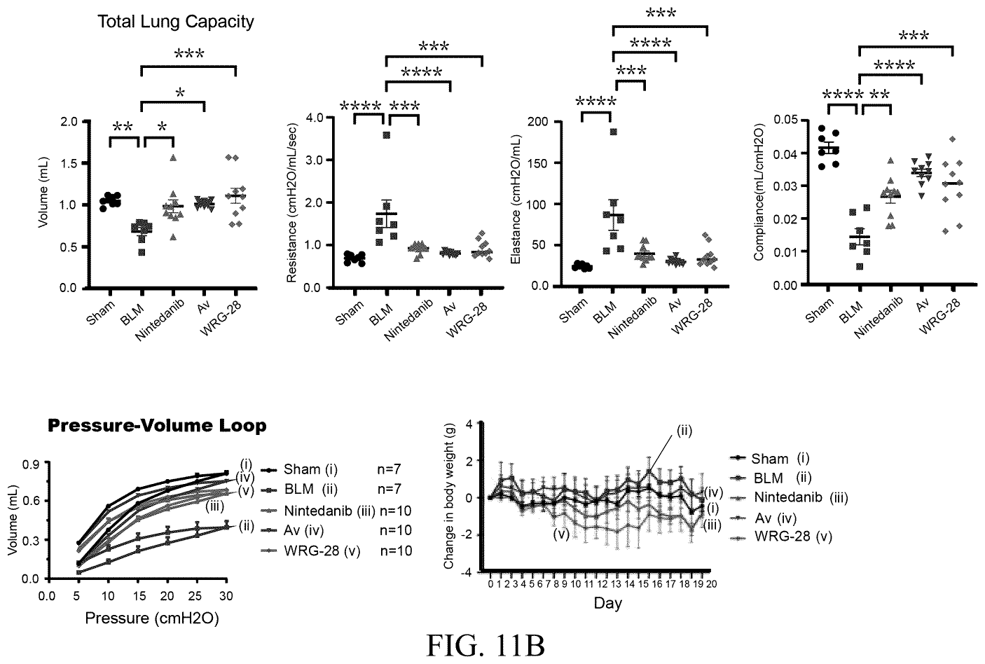

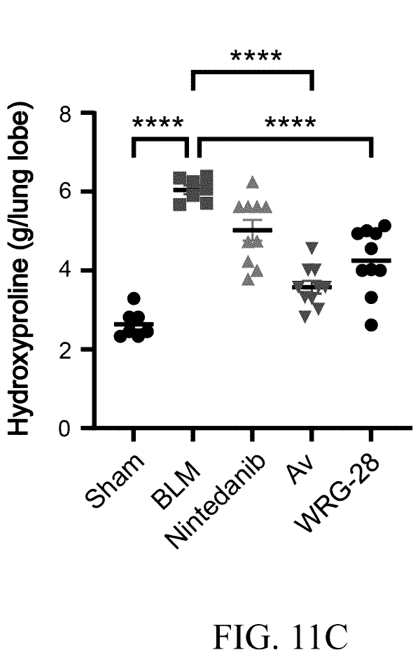

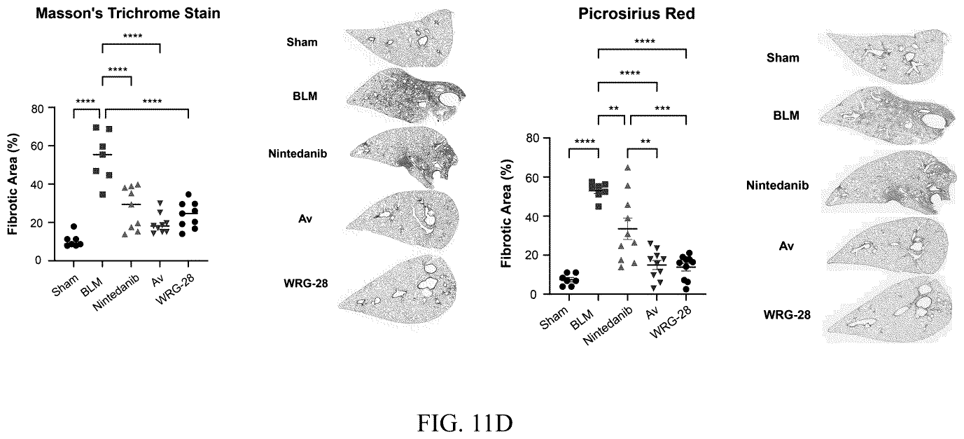

The efficacy of WRG-28 and Av in treating pulmonary fibrosis was examined in vivo using a BLM-induced pulmonary fibrosis mouse model for evaluation. 50 μL of Bleomycin (BLM, 3 mg/kg) or PBS were introduced by intratracheal instillation on day 0 to induce fibrosis or serve as the control group, respectively. This was followed by the administration of various compounds for 14 days (Nintedanib: 60 mg/kg/day, WRG-28:5 mg/kg/day, Av: 5 mg/kg/day). Lung function evaluation was conducted on day 21 (FIG. 11A). Nintedanib, an FDA-approved drug for idiopathic pulmonary fibrosis (IPF) treatment, was used as the positive control. The results of the lung function evaluation are depicted in FIG. 11B. These results indicate that the administration of BLM leads to a decrease in lung capacity and compliance, while increasing lung resistance and elastance. The degree of these changes can be reversed through treatment with Nintedanib, WRG-28 or Av. A marker of collagen deposition, hydroxyproline content, was extracted and analyzed to demonstrate the degree of fibrosis, as shown in FIG. 11C. BLM can effectively induce pulmonary fibrosis, while the administration of Nintedanib, Av or WRG-28 inhibits the progression of fibrosis. Histological analysis was conducted through staining techniques to quantify the fibrotic area. Both Masson's trichrome and Picrosirius Red staining demonstrate that treatment with Nintedanib, Av or WRG-28 effectively reduces the fibrotic area (FIG. 11D).

Those skilled in the art will readily observe that numerous modifications and alterations of the embodiments may be made while retaining the teachings of the present disclosure. Accordingly, the above disclosure should be construed as limited only by the metes and bounds of the appended claims.

What is claimed is:

1. A method for treating or preventing a CRAC (calcium release-activated calcium) channel-related disorder or condition and/or DDR2-related disorder or condition, comprising administering a subject in need thereof with a pharmaceutical composition comprising:
an effective amount of at least one selected from the group consisting of WRG-28, atovaquone, WRG-28 precursors, and atovaquone precursors; and
pharmaceutically acceptable carriers thereof.

2. The method of claim 1, wherein the CRAC channel-related disorder or condition and/or the DDR2-related disorder or condition is selected from the group consisting of a cytokine storm syndrome, a fibrotic disorder, cancer, a cardiorespiratory disease, an inflammatory disease, an autoimmune disease, an allergic disease, an acute kidney injury, a chronic kidney disease, a uremic cardiomyopathy, a polycystic kidney disease, and any combination thereof.

3. The method of claim 2, wherein the CRAC channel-related disorder or condition and/or the DDR2-related disorder or condition is the cytokine storm syndrome.

4. The method of claim 3, wherein the cytokine storm syndrome is an infection-induced cytokine storm syndrome.

5. The method of claim 4, wherein the cytokine is selected from the group consisting of a chemokine, an interferon, an interleukin, a lymphokine, and a tumor necrosis factor.

6. The method of claim 5, wherein the cytokine is an interleukin or a tumor necrosis factor.

7. The method of claim 2, wherein the CRAC channel-related disorder or condition and/or the DDR2-related disorder or condition is the fibrotic disorder.

8. The method of claim 7, wherein the fibrotic disorder is selected from the group consisting of cardiac fibrosis, pulmonary fibrosis, liver fibrosis, renal fibrosis, and any combination thereof.

9. The method of claim 7, wherein the fibrotic disorder is selected from the group consisting of an infection-induced fibrotic disorder, an obstruction-induced fibrotic disorder, drug-induced fibrosis, inflammatory-induced fibrotic disorder, and any combination thereof.

10. The method of claim 7, thereby improving a kidney function, a pulmonary function, a liver function, and/or a cardiac function; promoting a tissue repair and/or an epithelium differentiation; and/or inhibiting a collagen deposition, a myofibroblast expansion, and/or a TGF-β-associated fibroblast activation.

11. The method of claim 2, wherein the fibrotic disorder is nephrogenic systemic fibrosis or cystic fibrosis, and the inflammatory disease is arthritis or inflammatory bowel disease.

12. The method of claim 10, wherein the TGF-β-associated fibroblast activation is a TGF-β1-associated fibroblast activation.

13. The method of claim 1, the WRG-28, the atovaquone, the WRG-28 precursors, or the atovaquone precursors inhibit CRAC channel activation, DDR2 activation, store-operated Ca²⁺ entry, and/or cytokine expression.

14. The method of claim 13, wherein the cytokine is selected from the group consisting of any one of interleukin 1 to interleukin 36, a tumor necrosis factor alpha, a tumor necrosis factor beta, a CD40 ligand, a Fas ligand, a tumor necrosis factor-related apoptosis inducing ligand, and a tumor necrosis factor superfamily member 14, and any combination thereof.

15. The method of claim 14, wherein the cytokine is an interleukin 2, an interleukin 6, or the tumor necrosis factor alpha.

16. A method for inhibiting CRAC (calcium release-activated calcium) channel activation and/or DDR2 activation in a cell of a subject, comprising administering the subject in need thereof with a pharmaceutical composition comprising:
an effective amount of at least one selected from the group consisting of WRG-28, atovaquone, WRG-28 precursors, and atovaquone precursors; and
pharmaceutically acceptable carriers thereof.
